(19) United States
(12) Patent Application Publication
(10) Pub. No.: US 2007/0286886 A1
(43) Pub. Date: Dec. 13, 2007

(54) Materials And Methods For Inducing Apoptosis In Adipocytes

(75) Inventors: Michael B. Zemel, Knoxville, TN (US); Xiaocun Sun, Knoxville, TN (US)

Correspondence Address: VENABLE LLP, P.O. BOX 34385, WASHINGTON, DC, 20043-9998, US

(73) Assignee: University of Tennessee Research Foundation, Knoxville, TN (US)

(21) Appl. No.: 11/665,582

(22) PCT Filed: Mar. 29, 2005
(86) PCT No.: PCT/US05/10710

§ 371 Date: Apr. 16, 2007

Related U.S. Application Data
(60) Provisional application No. 60/618,595, filed on Oct. 15, 2004.

(51) Int. Cl. A61K 33/06; A61K 33/10; A61K 35/20; A61K 47/00; A61K 9/14; A61P 3/04

(52) U.S. Cl. 424/439; 424/489; 424/535; 424/682; 424/687

(57) ABSTRACT

The subject invention provides for materials and methods of using dietary calcium, calcium-containing products, dairy and antagonists of calcitrophic hormone activity for inducing apoptosis in adipocytes in order to reduce the number of adipocytes in an individual regulating body weight.

### FIELD OF INVENTION

The present invention relates to materials and methods for using dietary calcium, calcium-containing products, dairy, and antagonists of calcitrophic (1α,25-dihydroxyvitamin D_{3[}1α,25-(OH)₂-D₃]) hormone activity for inducing apoptosis in adipocytes in order to reduce the number of adipocytes in an individual regulating body weight.

### BACKGROUND OF THE INVENTION

Obesity and weight gain are characterized by an increase in both adipocyte size and number. Adipose tissue is thus linked to the dynamic role played by adipocytes. It has been hypothesized that the only effective method of reducing the number of adipocytes in an individual is through invasive surgical techniques such as liposuction.

The inventors had previously discovered that dietary calcium, calcium-containing products, dairy and antagonists of calcitrophic hormone activity have had a role in treating obesity and attenuating weight management, U.S. Pat. No. 6,384,087 and U.S. Patent Application Publication No. 20020192264.

The concept of adipocyte deletion by apoptosis is relatively recent. Recent evidence has shown that rat adipocytes underwent apoptosis following brain administration of leptin (Qian, H., Azain, M. J., Compton, M. M., Hartzell, D. L., Hausman, G. J., and Baile, C. A. (1998), Brain administration of leptin causes deletion of adipocytes by apoptosis, Endocrinology 139, 791-794). A depot-specific susceptibility to apoptosis in human preadipocytes has been found. (Niesler, C. U., Siddle, K., and Prins, J. B. (1998), Human preadipocytes display a depot-specific susceptibility to apoptosis, (Diabetes 47, 1365-1368).

Conjugated linoleic acid (CLA) reduces body fat content in part by inducing adipocyte apoptosis, and some factors, including PPARs, UCPs, leptin, and TNF-α, are involved (McCarty, M. F. 2000, Activation of PPARgamma may mediate a portion of the anticancer activity of conjugated linoleic acid, Med. Hypotheses 55, 187-188; Tsuboyama-Kasaoka, N., Takahashi, M., Tanemura, K., Kim, H. J., Tange, T., Okuyama, H., Kasai, M., Ikemoto, S., and Ezaki, O. (2000), Conjugated linoleic acid supplementation reduces adipose tissue by apoptosis and develops lipodystrophy in mice, Diabetes 49, 1534-1542; and Wright, S. C., Zheng, H., Zhong, J., Torti, F. M., Larrick, J. W. (1993), Role of protein phosphorylation in TNF-induced apoptosis: phosphatase inhibitors synergize with TNF to activate DNA fragmentation in normal as well as TNF-resistant U937 variants, J. Cell Biochem. 53, 222-233).

There is a need to provide non-invasive and non-toxic methods for reducing the number of and inducing apoptosis in adipocytes, without necrosis or scarring, for treating obesity and losing and maintaining healthy normal body weight. However, the data on the mechanisms and regulation of adipocyte apoptosis are still limited.

### SUMMARY OF THE INVENTION

The present invention relates to materials and methods for using dietary calcium, calcium-containing products, dairy and antagonists of calcitrophic (1α,25-dihydroxyvitamin D₃ (“1α,25-(OH)₂-D₃”)) hormone activity for inducing apoptosis in adipocytes in order to reduce the number of adipocytes in an individual regulating body weight.

### BRIEF DESCRIPTION OF THE DRAWINGS

FIG. 1A shows caspase-1 expression in 3T3 L1- and UCP2-transfected 3T3 L1 adipocytes. Primary-cultured adipocytes were treated with or without 1α,25-(OH)₂-D₃ (0.1 nM, 1 nM, 5 nM, 10 mM, and 100 nM) and dinitrophenol (DNP, 25 μM and 50 μM; DNP is used as a positive control for the effects of metabolic uncoupling) for 24 h for mRNA levels. Caspase-1 mRNA levels were measured by real-time PCR. Data are expressed as means ±se (n=6). Different letters above the bars indicate a significant difference at level of P<0.05. FIG. 1B shows caspase-3 expression in 3T3 L1- and UCP2-transfected 3T3 L1 adipocytes. Primary-cultured adipocytes were treated with or without 1α,25-(OH)₂-D₃ (0.1 nM, 1 mM, 5 nM, 10 nM, and 100 nM) and DNP (25 μM and 50 μM) for 24 h for mRNA levels. Caspase-1 mRNA levels were measured by real-time PCR. Data are expressed as mean±se (n=6). Different letters above the bars indicate a significant difference at level of P<0.05. FIG. 1C shows Bcl-2/Bax expression ratio in 3T3 L1- and UCP2-transfected 3T3 L1 adipocytes. Primary-cultured adipocytes were treated with or without 1α,25-(OH)₂-D₃ (0.1 nM, 1 nM, 10 nM, and 100 nM) and DNP (25 μM and 50 μM) for 24 h for mRNA levels. Caspase-1 mRNA levels were measured by real-time PCR. Data are expressed as mean±se (n=6). Different letters above the bars indicate a significant difference at level of P<0.05.

FIGS. 2A, B and C show caspase-1 and caspase-3 expression and Bcl-2/Bax ratio, respectively, in white adipose tissue of aP2-agouti transgenic mice treated with low calcium diets to increase 1α,25-(OH)₂-D₃ levels or high calcium diets to suppress 1α,25-(OH)₂-D₃ levels. Data are expressed as mean±se (n=8). Different letters above the bars indicate a significant difference at level of P<0.05.

FIG. 3 shows mitochondrial potential in 3T3 L1- and UCP2-transfected 3T3 L1. Primary-cultured adipocytes were treated with or without 1α,25-(OH)₂-D₃ (0.1 nM, 1 nM, 5 nM, 10 μM, and 100 nM) for 4 h before assay. Mitochondrial potential was measured using spectrofluorometry. Data are expressed as mean±se (n=6). Different letters above the bars indicate a significant difference at level of P<0.05.

FIG. 4 shows ATP production in 3T3 L1- and UCP2-transfected 3T3 L1. Primary-cultured adipocytes were treated with or without 11; 25-(OH)₂-D₃ (1 nM, 5 nM, 10 nM, and 100 nM) for 4 h before assay. ATP levels were measured using a microplate luminometer. Data are expressed as mean±se (n=−6). Different letters above the bars indicate a significant difference at level of P<0.05.

FIG. 5 shows intracellular calcium levels in 3T3 L1- and UCP2-transfected 3T3 L1 adipocytes. Primary-cultured adipocytes were treated with or without 1α,25-(OH)₂-D₃ (1 nM, 5 nM, 10 nM, and 100 nM) immediately before assay. Intercellular calcium levels were measured using a dual-wavelength fluorescence microscope. Data are expressed as mean±se (n=6). Different letters above the bars indicate a significant difference at level of P<0.05.

FIG. 6 shows mitochondrial calcium levels in 3T3 L1- and UCP2-transfected 3T3 L1 adipocytes. Primary-cultured adipocytes were treated with or without 1α,25-(OH)₂-D₃ (1 nM, 5 nM, 10 nM, and 100 nM) instantly before assay. Mitochondrial calcium levels were measured using a dual-wavelength fluorescence microscope. Data are expressed as mean±se (n=6).

FIG. 7 depicts a schematic illustration of the effects and mechanisms of 1α,25-(OH)₂-D₃ on adipocyte apoptosis. Physiological low doses of 1α,25-(OH)₂-D₃ restore mitochondrial potential and ATP production by suppressing UCP2, thereby inhibiting apoptosis. By contrast, a high dose of 1α,25-(OH)₂-D₃ induces mitochondrial calcium overload and stimulates apoptosis.

FIG. 8 represents a western blot with anti-mouse UCP2 antibody of cell lysates harvested 48 hrs after siRNA transfection for evaluation of UCP2 protein reduction (upper panel). The internal control actin is shown in the low panel. Blot shown is representative of three similar experiments.

FIG. 9 shows UCP2/Actin expression in siRNA transfected 3T3-L1 (UCP2 ko v. control).

FIG. 10 shows mitochondrial potential in 3T3-L1 and siRNA transfected 3T3-L1(UCP2 ko). Data are expressed as mean±SE (n=6). *P<0.05 vs. control.

FIG. 11 shows caspase-3 expression in 3T3-L1 and siRNA transfected 3T3-L1 (UCP2 ko) cells. Data are expressed as mean±SE (n=6). *P<0.01 vs. control.

FIG. 12 shows the effect of mitochondrial uncoupling inhibitor GDP on mitochondrial potential in 3T3-L1 cells. Data are expressed as mean±SE (n=6). *P<0.05 vs. control.

FIG. 13 shows the effect of mitochondrial uncoupling inhibitor GDP on caspase-3 expression in 3T3-L1 cells. Data are expressed as mean±SE (n=6). *P<0.01 vs. control, *P<0.01 vs. 100 μM GDP.

FIG. 14 shows the effect of calcium channel ionophore BK 8644 on intracellular calcium levels in 3T3-L1 cells. Chemical treatment was conducted. Data are expressed as mean±SE (n=6). *P<0.05 vs. 1 nM Bay K 8644, **P<0.05 vs. 5 nM Bay K 8644, ***P<0.01 vs. 10 nM Bay K 8644, ****P<0.01 vs. 50 nM Bay K 8644.

FIG. 15 shows the effect of calcium channel ionophore Bay K 8644 on caspase-3 expression in 3T3-L1 cells. Data are expressed as mean±SE (n=6). *P<0.01 vs. control, **P<0.05 vs. 5 nM and 10 nM Bay K 8644, ***P<0.01 vs. 50 nM Bay K 8644.

FIG. 16 shows the effect of mitochondrial uncoupling inhibitor GDP and calcium channel ionophore BK 8644 on mitochondrial potential in 3T3-L1 cells. Data are expressed as mean±SE (n=6). *P<0.05 vs. control.

FIG. 17 shows the effect of mitochondrial uncoupling inhibitor GDP and calcium channel ionophore BK 8644 on caspase-3 expression in 3T3-L1 cells. Data are expressed as mean±SE (n=6). *P<0.01 vs. control, ** p<0.01 vs. control and 1 nM Bay K 8644, ***p<0.05 vs. 1 mM GDP.

FIG. 18 shows the effect of calcium channel ionophore Bay K 8644 on mitochondrial calcium in 3T3-L1 cells. Data are expressed as mean±SE (n=6).

### DETAILED DISCLOSURE OF THE INVENTION

The subject invention relates to materials and methods for using dietary calcium, calcium-containing products, dairy products and antagonists of calcitrophic hormone activity for inducing apoptosis in adipocytes in order to reduce the number of adipocytes in an individual regulating body weight. By reducing the number of adipocytes, an individual is less able to store excess energy coming from rebound in food intake, after stopping a weight loss diet. Also, generating new fat cells requires extra energy that contributes to a further metabolic enhancement favoring lean body mass.

In an exemplary embodiment of the invention, a calcium-containing product is administered in an amount effective to induce apoptosis of adipocytes in an individual in order to reduce the amount or number of adipocytes in said individual. The inventive method provides an alternative manner of reducing or maintaining the amount of adipocyte cells without resorting to drastic or invasive surgical techniques, such as liposuction. The invention is directed to individuals desiring, intending, or in need of reduction of the amount of adipocytes in said individual in order to maintain a normal healthy body weight or treating or avoiding weight related conditions, such as obesity.

In an exemplary embodiment, the subject invention provides methods of inducing apoptosis in adipocytes by increasing the ingestion of calcium. In an exemplary aspect, the calcium is contained in a calcium-containing product. Examples of calcium-containing products include dietary calcium, calcium carbonate, dairy or a product derived from dairy, such as milk, yogurt or cheese, a whey-derived protein calcium-containing product that is derived from a product containing whey protein, such as milk, cream or cheese whey. The calcium-containing product may be, e.g., yogurt or a product derived from yogurt, cheese or a product derived from cheese, or milk or a product derived from milk, such as skim milk, 1% milk, 2% milk, whole milk, half and half or whipping cream. In another exemplary embodiment, the calcium-containing product is in the form of a powder or is incorporated into a nutritional or dietary composition or supplement or calcium fortified vitamin supplements, or is incorporated into a food product or foodstuff or other foods high in calcium or any food product that is consumed by the individual. The food product may be a beverage or a liquid supplemented with calcium, such as acidic juice beverages, acidic beverages, neutral pH beverages, nutritional supplement foodstuffs, confectionery products, dairy products, non-dairy products naturally high in calcium, food or food stuff fortified with calcium, bakery products and farinaceous products, or orange juice, apple juice, grape juice, grapefruit juice, cranberry juice, blended juice, milk, soy milk, shake, smoothie, frappe, high-energy protein bar, high calcium chews, chewing gum, chocolate, or cookie, yogurt, ice cream, cheese, processed cheese, bread, muffin, biscuit, cereal or roll. The calcium may be contained in cereal, salmon, beans, tofu, spinach, turnip greens, kale, broccoli, waffles, pancakes, pizza, cottage cheese, ice cream or frozen yogurt. In another exemplary embodiment, the calcium-containing product comprises infant formula, nutriceuticals, or meal replacement beverages or drinks.

In another embodiment, the product may be in the form of a pill, tablet, capsule, or combination with other minerals and/or vitamins. The product may be for human or non-human/animal consumption, such as pet food or farm/agricultural animal feed.

The present invention also provides methods of increasing the amount of calcium ingested or consumed by the individual and, optionally, in combination with other dietary efforts, e.g., restricting the caloric intake or exercising, and/or administering the calcium-containing product for a prolonged period of time to obtain the desired results of adipocyte apoptosis and reduction thereof. For example, the product may be administered for a continuous interval of at least about one week, two weeks, three weeks, one month, six weeks, two months, three months, six months, or one year, wherein the product is administered on an average daily basis in amounts effective to induce apoptosis in adipocytes. The amount effective to induce apoptosis in adipocytes may be based on the amount of dietary calcium contained in said product, e.g., at least about 1000 mg calcium per day. A calcium-containing product or a serving thereof may contain on average at least about 100 mg, 200 mg, 255 mg, 300 mg, 400 mg or 500 mg. Dairy calcium consumption may be at least about 500 mg, 600 mg, 773 mg, 800 mg, 900, mg, 1000 mg, 1100 mg, 1200 mg, 1300 mg, 1346 mg, 1400 mg, 1500 mg, 1600 mg, 1700 mg 1800 mg, 1900 mg or 2000 mg. In an exemplary embodiment, the calcium-containing product is administered over a continuous interval of at least about 1000 mg, 1300 mg or 1400 mg on an average daily basis. These dosages may be referred as being “high calcium”.

Another aspect of the invention provides for methods of determining dietary calcium consumption of the individual, wherein the individual is a human and (1) if the dietary calcium consumption is below 1000 mg/day, increasing the dietary calcium consumption, and (2) if the dietary calcium consumption is at least about 1000 mg/day, maintaining the dietary calcium consumption. In another embodiment the individual is, e.g., a human and the amount of dietary calcium consumed by the individual before administering the effective amount of calcium-containing products is less than about 400 mg/day, 600 mg/day or 773 mg/day. In another aspect, the individual may be a human and the average daily calcium administered may be at least about 1000 mg/day, 1100 mg/day, 1200 mg/day, 1346 mg/day, or 1400 mg/day.

In yet another exemplary embodiment, the effective amount of calcium-containing product may be in the form of a dairy product which is administered in at least about 3 to 4 servings per day, e.g., 3 or 3.5 servings. An example of serving size may comprise at least about 8 ounces of milk, 8 ounces of yogurt or 1.5 ounces of cheese. In an exemplary embodiment, a serving may contain at least about 200, 255 or 300 mg, two servings may contain about 700, 773 or 800 mg, and three servings may contain 1000, 1100, 1300, 1346 or 1400 mg. In one aspect, the serving portion may contain on average, e.g., for 8 ounces of milk at least about 300 mg of dietary calcium, for 8 ounces of yogurt at least about 300 mg of dietary calcium or for 1.5 ounces of cheese at least about 300 mg of dietary calcium. In another aspect of the invention, a serving portion may contain on average, e.g., for 6 ounces of yogurt at least about 200 mg of dietary calcium. In an exemplary embodiment, the effective amount of dietary calcium is at least about 1000 mg per day. In another embodiment, the calcium-containing product may be administered on an average daily basis equaling to at least about 50 to 75 servings per month or 100 to 120 servings per month. In a recommended embodiment, the amount of calcium is administered on average of about 1346 mg a day, or 3 to 3.5 servings of dairy to achieve the desired effects of inducing adipocyte apoptosis and losing weight.

The term “apoptosis” is used to describe the morphological changes that characterize cells undergoing programmed cell death. Apoptotic cells have a shrunken appearance with altered membrane lipid content and highly condensed nuclei. Apoptotic cells are rapidly phagocytosed by neighboring cells or macrophages without leaking their potentially damaging contents into the surrounding tissue. As used herein, various terms may be used synonymously, such as causes or induces apoptosis of, kills, reduces, depletes, destroys, exterminates, annihilates, eliminates excess, increases breakdown of, promotes loss of, causes programmed cell death of adipocytes or adipose or fat cells.

The term “adipocyte” is one that is generally recognized in the art, e.g., referring to an adipose cell or a fat cell, terms which can be interchangeable, and which refer to one of the fat-laden cells making up adipose tissue. The invention is directed to using calcium-containing products to modulate calcium signaling and intracellular calcium concentrations ([Ca²⁺]i), as well as the expression of uncoupling protein 2 (UCP2) in order to induce apoptosis in adipocytes.

As used herein, Ca²⁺ is a ubiquitous intracellular messenger involved in many cellular processes. To generate such complex Ca²⁺ signals, cells rely on the rapid release of the Ca²⁺ storage (such as the endoplasmic reticulum (“ER”) and sarcoplasmic reticulum (“SR”) and mitochondria) as well as on the controlled Ca²⁺ influx from the extracellular medium upon stimuli, and strive to maintain the Ca²⁺ concentration at extremely low levels by expelling Ca²⁺ ions to the exterior and by compartmentalization of Ca²⁺ intracellular stores under physiological conditions of rest. Mitochondria take up Ca²⁺ through the uniporter on the inner membrane at the expense of Δψ (the electrical potential gradient across the mitochondrial membrane). Mitochondria are often located close to the ER and therefore are exposed to the Ca²⁺ release by the inositol-1,4,5-trisphosphate receptor (IP3R) and ryanodine receptor (RyR). The high Ca²⁺ levels achieved at these contact sites favor Ca²⁺ uptake into mitochondria. Because of their tight coupling to ER Ca²⁺ store, mitochondria are highly susceptible to abnormalities in Ca²⁺ signaling. The amount of Ca²⁺ going through mitochondria is crucial in triggering a Ca²⁺-dependent apoptosis response, possibly by the opening of a sensitized state of permeability transition pore (PTP).

In yet another aspect, the invention relates to the regulation of one or more of the functional groups of molecules involved in apoptosis, e.g., caspases and the Bcl-2 family, or others that are modulated by [Ca²⁺]i such as proteases, e.g., caspase-3, caspase-9, and Ca²⁺-dependent endonucleases. The invention may be used to modulate the interaction that occurs between Bcl-2 family proteins and calcium signaling in the execution of apoptosis, e.g., BAX and BAK which can play a critical role in maintaining of homeostatic concentration of [Ca²⁺] in endoplasmic reticulum (ER) and mitochondria, which can control the apoptotic fate of cells responding to [Ca²⁺]i-dependent stimuli.

In another aspect of the invention, an individual increases the consumption of dietary calcium, calcium-containing products, dairy, antagonists of calcitrophic hormone activity to inhibit 1α,25-(OH)₂-D₃ to modulate [Ca²⁺]i and the calcium signaling process in order to induce apoptosis in adipocytes.

1α,25-(OH)₂-D₃ can modulate adipocyte lipid and energy metabolism via both genomic and nongenomic mechanisms by modulating adipocyte Ca²⁺ signaling, resulting in increased lipogenesis and decreased lipolysis. In addition, 1α,25-(OH)₂-D₃ also can play a role in regulating human adipocyte mitochondrial uncoupling protein 2 (UCP2) mRNA and protein levels, indicating that the suppression of 1α,25-(OH)₂-D₃ and the resulting up-regulation of UCP2 may contribute to increased rates of lipid oxidation as well as to a decrease in mitochondrialΔψ, thereby causing a further increase in apoptosis.

In another embodiment, the instant invention provides a method comprising administering an antagonist of calcitrophic hormone (1α,25-(OH)₂-D₃) activity in an amount effective to block calcitrophic hormone activity to induce apoptosis in adipocytes in an individual in order to reduce the amount or number of adipocytes in said individual. The antagonist may be a 1α,25-(OH)₂-D₃ receptor antagonist, such as an antibody that binds to said 1α,25-(OH)₂-D₃ receptor or a chemical compound that binds to said 1α,25-(OH)₂-D₃ receptor, or an analog, homolog or isomer of 1α,25-(OH)₂-D₃ that binds to the 1α,25-(OH)₂-D₃ receptor and antagonizes the function of the receptor. An exemplary antagonist is 1-β,25, dihydroxyvitamin D₃.

In another aspect of the invention, the antagonist may be a 1α,25-(OH)₂-D₃ antagonist selected from the group consisting of an antibody that binds to said 1α,25-(OH)₂-D₃, a chemical compound that binds to said 1α,25-(OH)₂-D₃, one or more soluble 1α,25-(OH)₂-D₃ receptors, 1α,25-(OH)₂-D₃ neutralizing antibodies; soluble 1α,25-(OH)₂-D₃ receptor; fusion proteins comprising the 1α,25-(OH)₂-D₃ receptor; or compounds comprising calcium. In yet another embodiment, the antagonist of calcitrophic hormone activity may be dietary calcium, calcium-containing products or dairy.

Fusion proteins comprising the 1α,25-(OH)₂-D₃ receptor may be made according to methods known in the art. An exemplary type of fusion protein comprises the soluble form of the 1α,25-(OH)₂-D₃ receptor fused to Ig heavy chains according to the teachings of Capon et al. (U.S. Pat. Nos. 5,565,3375 and 5,336,603, hereby incorporated by reference in their entireties). In other embodiments, at least one 1α,25-(OH)₂-D₃ receptor (Nemere, et al., J. Bone Miner. Res. (1998) 13:1353-59; Fleet, et al., Nutr. Rev. (1999) 57:60-64) is incorporated into liposomes which are preferentially targeted to adipocytes using, for example, acylation stimulating protein (ASP) (Kalant et al., Clin. Invest. Med. (1995) 18 (Supp. B:B10); Maslowska, et al., Int. J. Obesity (1998) 22:S108; Maslowska, et al., Acylation Stimulating Protein (ASP): Role in Adipose Tissue, in Progress in Obesity Research: 8, (1999), Ed. B. Gut-Grand and G. Ailhaud, John Libbey & Co.). In other embodiments, soluble forms of the 1α,25-(OH)₂-D₃ receptor may be coupled to adipocyte targeting agents such as the ASP. Soluble forms of the 1α,25-(OH)₂-D₃ receptor may be produced from the membrane bound form of the Vitamin D receptor according to methods known in the art. Coupling of one or more soluble 1α,25-(OH)₂-D₃ receptors to one or more adipocyte targeting agents may be accomplished recombinantly or using chemical crosslinking compounds, such as those sold by Pierce (Rockford, Ill.).

A calcitrophic hormone antagonist of the invention which acts as an antagonist of calcitrophic hormone activity includes those set forth above. Without wishing to be bound by any particular mechanism, it is suggested that the inventive antagonists of calcitrophic hormone activity can inhibit the interaction between the ligand and receptor, for example by binding to the receptor or to the ligand, and/or by blocking access of the receptor by its agonists through steric hindrance on the cell membrane or by interfering with the associated signaling process.

The instant invention may include an antagonist which blocks the action of 1α,25-(OH)₂-D₃ in adipocytes or whereby the administering of said antagonist decreases the levels of calcitrophic hormones in the adipocytes. Calcitrophic hormone activity in adipocytes that is blocked may include one or more of the following: inhibiting apoptosis, inhibiting lipolysis, stimulating lipogenesis, increasing adiposity, stimulating triglyceride accumulation, increasing intracellular calcium concentration ([Ca²⁺]i), inhibiting adipocyte uncoupling protein 2 (UCP2) expression and/or stimulating fatty acid synthase (FAS) activity. The administered antagonist may suppress adiposity, inhibit triglyceride accumulation, reduce, suppress or decrease intracellular calcium concentration ([Ca²⁺+]i), increases adipocyte uncoupling protein 2 (UCP2) expression, increase core temperature, accelerate weight loss and fat mass reduction in an individual under caloric restriction, and/or prevent stimulation of fatty acid synthase (FAS) activity. In one aspect of the invention, the antagonist may suppress adiposity and inhibit triglyceride accumulation by stimulating lipolysis and inhibiting lipogenesis or may induce a metabolic state in which the energy metabolism is shifted from energy storage to energy expenditure.

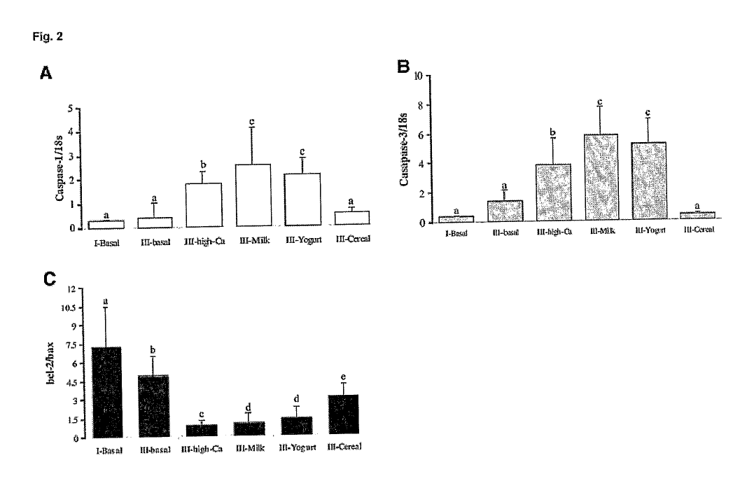

As detailed in Example 1, the notion that an increase of the Bcl-2/Bax expression ratio indicates a protective role of lower level of 1α,25-(OH)₂-D₃ against apoptosis, and lower ratio of Bcl-2/Bax indicates a decrease in protection from apoptotic death. FIG. 2C of Example 1 shows that mice fed with high calcium, milk and yoghurt resulted in a Bcl-2/Bax expression ratio of about 1.0 to 1.5 and mice fed with low calcium had a Bcl-2/Bax expression ratio of about 5.0 to 7.5. Therefore, inventive methods of administering calcium-containing product are from about 66% to about 86% more effective in inducing apoptosis than methods using low or no calcium.

High calcium diets reduce adipocyte numbers in the truncal region by at least about 10% or 20% to 30%. This can be measured in terms of adipocyte number per unit volume or per unit weight of fat tissue. Adipocyte number can be measured by assaying total DNA in a unit of fat tissue, which is directly proportional to the number of cells. According to the inventive methods, a high calcium diet reduces the volume of fat, not just by reducing the volume of adipocytes, but by killing about 20-30% of the adipocytes in a given volume of fat tissue. Thus, the reduction in truncal fat may result from reductions both in adipocyte numbers and average size.

A method for treating a subject in need of such treatment (e.g., a human patient or other animal subject having a condition or disease that is mediated by a calcitrophic hormone antagonist), comprising administering to the subject an effective amount of an agent of the invention, e.g., an antagonist of calitrophic hormone activity. Such a method can, e.g., treat, prevent, ameliorate, control, suppress, stop, slow and/or inhibit the condition. The invention may be carried out using a pharmaceutical composition, which comprises an agent of the invention (e.g., a therapeutically effective amount of the peptide) and a pharmaceutically acceptable carrier.

In one embodiment of the invention, an antagonist of calcitrophic hormone activity is administered in various doses to stimulate apoptosis in adipocytes. The administration of the antagonist of calcitrophic hormone activity may be performed in vivo or in vitro, e.g., in isolated human or animal adipocytes, e.g., in 3T3-L1 and L1-UCP2 cells, or in humans or animals, e.g., in aP2 transgenic mice. The antagonist of calcitrophic hormone activity may stimulate apoptosis via a calcium-dependent mechanism or by antagonizing calcitrophic hormone activity, e.g., by increasing mitochondrial Ca²⁺.

Increasing dietary calcium, calcium-containing products, dairy or antagonists of calcitrophic hormone activity may be used to suppress 1α,25-(OH)₂-D₃ thereby attenuating adipocyte triglyceride accumulation and causing a net reduction in fat mass in either humans or non-human animals, such as mice, in the absence of caloric restriction, a marked augmentation of body weight and fat loss during energy restriction in both mice and humans, and a reduction in the rate of weight and fat regain after food restriction in mice.

The invention may relate to using high calcium diets to suppress 1α,25-(OH)₂-D₃ stimulate adipose tissue apoptosis and contribute to weight management and anti-obesity effects. High-calcium diets also attenuate the decreases in core temperature that otherwise occur with energy restriction and up-regulate UCP2 expression in white adipose tissue.

The effects of dietary calcium, calcium-containing products, dairy or antagonists of calcitrophic hormone activity may be due to a loss of adipocytes which result in a cellular deficit in lipid esterification as the body recovers from energy restriction, which may further lead to the regulation of body weight, which may also be due to both effects on lipolysis and lipogenesis.

In another embodiment of the invention, dietary calcium, calcium-containing products, dairy or antagonists of calcitrophic hormone activity may effect adipocyte apoptosis by effecting UCP2, mitochondrial uncoupling, and 1α,25-(OH)₂-D₃.

As set forth in Examples 1 and 2, the inventors demonstrate that by using high-calcium diets, including dietary calcium, calcium-containing products, dairy and/or antagonists of calcitrophic hormone activity, apoptosis in adipocyte can be achieved.

For example, as elaborated on in Example 1, 1α,25-(OH)₂-D₃ has dual effects on apoptotic death in adipocytes: physiological doses of 1α,25-(OH)₂-D₃ inhibit adipocyte apoptotic death while suppression of 1α,25-(OH)₂-D₃ using high calcium diets stimulates adipose apoptosis in mice. In contrast, pharmacological doses of 1α,25-(OH)₂-D₃ stimulate apoptosis. Moreover, 1α,25-(OH)₂-D₃ inhibits expression of mitochondrial uncoupling protein 2 (UCP2), which plays a key role in regulation of mitochondrial potential and apoptosis. Low doses of 1α,25-(OH)₂-D₃ appear to inhibit adipocyte apoptotic death by inhibiting UCP2 expression while high doses stimulate adipocyte apoptotic death via a calcium dependent mechanism.

Mitochondria play a pivotal role in the coordination, initiation, and execution of apoptotic cell death. Three general mechanisms are suggested: 1) disruption of oxidative phosphorylation ATP production, 2) regulation of the apoptotic proteases, and 3) alteration of cellular reduction-oxidation potential. UCP2, which is highly expressed in white adipose tissue, may function as a mitochondrial uncoupler of oxidative phosphorylation, and thus participates in reducing efficiency of ATP synthesis and stimulating apoptosis in adipocytes.

Overexpressing UCP2 in 3T3-L1 cells induces marked reductions in mitochondrial potential (Δψ) and ATP production (P<0.01), increase in the expression of caspases (P<0.05), and decrease in Bcl-2/Bax expression ratio (P<0.01). As shown in Example 1, physiological doses of 1α,25-(OH)₂-D₃ (0.1-10 nM) restored mitochondrial Δψ in L1-UCP2 cells and protected against UCP2 overexpression-induced apoptosis (P<0.01), whereas a high dose (100 nM) stimulated apoptosis in 3T3-L1 and L1-UCP2 cells (P<0.05). In addition, 1α,25-(OH)₂-D₃ stimulated cytosolic Ca²⁺ dose-dependently in both 3T3-L1 and L1-UCP2 cells. However, physiological doses suppressed mitochondrial Ca²⁺ levels by ˜50% whereas the high dose increased mitochondrial Ca²⁺ by 25% (P<0.05). This explains stimulation of apoptosis by the high dose of 1α,25-(OH)₂-D₃.

Moreover, 1α,25-(OH)₂-D₃ directly suppresses UCP2 expression in isolated human adipocytes, indicating that the up-regulation of UCP2 induced by dietary calcium may result from the loss of inhibition of UCP2 expression by 1α,25-(OH)₂-D₃.

Accordingly, it appears that 1) mitochondrial uncoupling induces apoptosis in differentiated 3T3-L1 cells; 2) low doses of 1α,25-(OH)₂-D₃ inhibit apoptosis in differentiated 3T3-L1 cells in a dose-dependent manner whereas high doses stimulate apoptosis; and 3) high-calcium diets, which suppress 1α,25-(OH)₂-D₃ levels in vivo, stimulate adipose tissue apoptosis in aP2 transgenic mice.

As shown in Example 2, Ca²⁺ ionophore Bay K 8644 (BK8644) induced 3-4 fold of increases in [Ca²⁺]i (within the response range to physiological low doses of 1α,25-(OH)₂-D₃ and dose-dependently stimulated caspase-3 expression by 94%-260% (p<0.01). In contrast, guanosine 5′-diphosphate (GDP), a potent inhibitor of mitochondrial uncoupling, decreased [Ca²⁺]i, and increased mitochondrial potential, and suppressed caspase-3 expression in a dose dependent manner (47%-80%, p<0.05). To address the independent effect of mitochondrial uncoupling, pretreatment of 3T3-L1 cells with BK 8644 prevented GDP-induced decreases in [Ca²⁺]i but preserved the effect on GDP stimulated mitochondrial potential. GDP suppressed caspase-3 expression by 45% (p<0.05) in BK8644 pretreated cells. Transfection of dsRNA specific for UCP2 mRNA into 3T3-L1 cells suppressed UCP2 expression by 70%, and caused a 52% increase in mitochondrial potential but a 58% decrease in caspase-3 expression (p<0.05), indicating a direct role of mitochondrial uncoupling in adipocyte apoptosis. These data further demonstrate that physiological doses of 1α,25-(OH)₂-D₃ inhibit apoptosis and this effect is attributable to the inhibitory effect on mitochondrial uncoupling. In contrast, high doses of 1α,25-(OH)₂-D₃ stimulate apoptosis via a calcium-dependent mechanism.

The present invention may be used by an individual intending or desiring to induce apoptosis in adipocytes in order to regulate body weight, induce weight and/or fat loss, prevent weight and/or fat gain, and/or increase the metabolic consumption of adipose tissue in the individual. The invention may be used to treat a body weight condition, such as overweight or obesity, e.g., Grade I, II or III obesity. Individuals using the invention may be moderately overweight, slightly overweight, or intent on maintaining a normal weight, or if the individual has lost weight and is preventing or reducing weight regain or after weight loss.

The present invention also relates to methods of inducing adipocyte apoptosis in an individual in need thereof treating, reducing or attenuating, or is at risk of, excess body weight and/or an excess of body fat or obesity associated health problems or disorders, such as coronary artery disease, osteoarthritis, ligament injuries, perineal dermatitis, cardiomyopathy, urologic syndrome, high blood pressure, stroke, kidney stones, colon cancer, breast cancer, head and neck tumors, premenstrual syndrome, postpartum depression, hypertensive disorders of pregnancy, diabetes, Type-2 diabetes, high serum insulin levels, diabetes mellitus, depression, asthma, inflammatory bowel disease, attention deficit disorder, migraine headaches, kidney disease, hypercholesterolemia, congestive heart failure, and immune deficiency.

The term “individual” includes animals of avian, mammalian, or reptilian origin. Mammalian species which benefit from the disclosed methods include, and are not limited to, apes, chimpanzees, orangutans, humans, monkeys; domesticated animals (pets) such as dogs, cats, guinea pigs, hamsters, Vietnamese pot-bellied pigs, rabbits, and ferrets; domesticated farm animals such as cows, buffalo, bison, horses, donkey, swine, sheep, and goats; exotic animals typically found in zoos, such as bear, lions, tigers, panthers, elephants, hippopotamus, rhinoceros, giraffes, antelopes, sloth, gazelles, zebras, wildebeests, prairie dogs, koala bears, kangaroo, opossums, raccoons, pandas, giant pandas, hyena, seals, sea lions, and elephant seals. Reptiles include, and are not limited to, alligators, crocodiles, turtles, tortoises, snakes, iguanas, and/or other lizards. Avian species include, and are not limited to, chickens, turkeys, pigeons, quail, parrots, macaws, dove, Guinea hens, lovebirds, parakeets, flamingos, eagles, hawks, falcons, condor, ostriches, peacocks, ducks, and swans.

In one embodiment the individual is a human or a non-human animal, such as a pet, farm animal or laboratory animal. The pet may be a dog, cat, bird, rabbit, or hamster. The animal may be a laboratory test animal, such as a mouse. The individual may be a human, such as a male or female adult, a child, a post partum woman or an individual who has lost weight as a result of a previous diet.

Also provided are novel and advantageous methods of restoring normal body fat ratios via apoptosis of adipocytes in women post partum. In one such embodiment, the methods are practiced by identifying an individual who has recently given birth to a child and increasing the amount of dietary calcium consumed by the individual.

Methods of preventing or reducing the regain of weight lost after an initial period of dieting are also provided by the instant invention. This method can be practiced by identifying an individual who has lost weight as a result of a previous diet and increasing the amount of dietary calcium consumed by the individual.

The subject invention also provides methods of reducing the risk of obesity in a child by increasing the amount of calcium consumed by the child. Calcium intake can be accomplished by, for example, increasing the intake by the child of dairy products or other products containing high levels of calcium.

The present invention may be a method of determining the apoptotic effects in adipocytes by dietary calcium, calcium-containing products, dairy or antagonists of calcitrophic hormone activity, using known in vitro and in vivo studies, e.g., such as those used and described in Examples 1 and 2, or other known methods, e.g., using isolated human or non-human animal cells or cultured cells, or transgenic non-human animals.

Each of the methods discussed above may further comprise restricting the caloric intake of an individual. Additionally, dietary products containing high levels of calcium may be provided to the individual in conjunction with a dietary plan. The dietary products may be provided to the individual on a regular or scheduled basis or on demand by the individual.

In an exemplary embodiment, the invention relates to a method of reducing the amount of adipocytes in an individual comprising the steps of: (a) providing the individual with information disclosing that consuming an effective amount of calcium-containing products is associated with inducing apoptosis in adipocytes, and (b) providing the individual with a dietary plan for consuming calcium containing products effective to induce apoptosis in adipocytes in said individual. The invention may further include determining consumption of calcium-containing products by said individual, and formulating a dietary plan for consuming products containing an effective amount of calcium-containing products. In another aspect, the invention may include preparing an analysis of the individual's dietary intake of calcium-containing products, monitoring the consumption of calcium-containing products of the individual and/or monitoring the weight of the individual. This may include obtaining information about the amounts and types of foods consumed by the individual, which may be done, e.g., by log or questionnaire in either electronic or paper format.

In yet another aspect, the information may be obtained by having the individual answer questions over the internet, and the information is analyzed by a computer after input of the data by the individual, and the information is compared to a database containing the nutritive values of the foods, and the nutritional composition of the diet of the individual is provided, including the amount of calcium-containing products consumed, and further comprising providing recommendations regarding increases in the amount of calcium-containing products consumed by the individual if the amount of calcium-containing products consumed is suboptimal.

The invention may include determining the weight and the height of the individual, and optionally, calculating the body mass index of the individual and comparing the body mass index of the individual to established norms, and providing the individual with information relating to the benefits of maintaining a normal weight. In addition, the invention may further include monitoring the consumption of calcium-containing products of the individual and/or monitoring the weight of the individual. In yet another aspect, the invention may be carried out over a communication network comprising inputting weight values on a web page and comparing the values with a database available on the Internet, and optionally, providing the individual with calcium-containing products. The communication network may be the Internet, an intranet, LAN, WAN, a real private network, or two or more computers connected electronically. This method may further provide requesting verification that the weight and height values inputted by the individual are correct.

The analysis of the individual's dietary intake may be performed by a computer after input of the data related to food consumption by the individual. The foods consumed by the individual, as well as the amounts, are compared to a database containing the nutritive values of the foods and the nutritional composition of the diet of the individual is provided. After analysis of the nutritional composition of the foods ingested by the individual, the amount of calcium consumed by the individual is provided. Recommendations regarding increases in the amount of calcium consumed by the individual, as well as sources of dietary calcium, may be provided from a database which compares the amount of calcium consumed with that found to optimize or induce apoptotic weight loss.

In some instances, the caloric intake of an individual may be unmodified and caloric intake may be ad lib. In other instances, it may be desirable to reduce the caloric intake of the individual as part of the dietary plan. In one embodiment, the range of caloric intake of the individual is based upon gender. Caloric intake reduction can range from about 200 to about 1200 kcal per day in relation to their normal intake, although higher reductions are possible. In an exemplary embodiment, the range of caloric intake reduction is about 300-1000 kcal per day, preferably about 500 kcal per day.

The weight/height ratio may be calculated by obtaining the weight of an individual in kilograms (kg) and dividing this value by the height of the individual in meters. Alternatively, the weight/height ratio of an individual may be obtained by multiplying the weight of the individual in pounds (lbs) by 703 and dividing this value by the square of the height of the individual (in inches (in)). These ratios are typically referred to as BMI. Thus, BMI=kg/m2 or BMI=(lbs.×703)/(in)2.

Where BMI is utilized as a measure of obesity, an individual is considered overweight when BMI values range between 25.0 and 29.9. Obesity is defined as BMI values greater than or equal to 30.0. The World Health Organization assigns BMI values as follows: 25.0-29.9, Grade I obesity (moderately overweight); 30-39.9, Grade II obesity (severely overweight); and 40.0 or greater, Grade III obesity (massive/morbid obesity). Using weight tables, obesity is classified as mild (20-40% overweight), moderate (41-100% overweight), and severe (>100%) overweight. Individuals 20% over ideal weight guidelines are considered obese. Individuals 1-19.9% over ideal weight are classified as overweight.

A further aspect of the subject invention provides methods for promoting good health by providing a product with calcium wherein the provision of the product is accompanied by information regarding the benefits of the consumption of calcium with respect to inducing apoptosis in adipocytes. For example, the invention may include a method comprising communicating to a potential consumer that consuming a calcium-containing product induces apoptosis in adipocytes, the communicating being by an entity having a commercial interest in the consumption of the product. The communicating may comprise providing information about the required suboptimal amounts of dietary calcium or about the amounts of calcium-containing product consumption required to induce apoptosis in adipocytes. The communicating may be by verbal communication, pamphlet distribution, print media, audio tapes, magnetic media, digital media, audiovisual media, billboards, advertising, newspapers, magazines, direct mailings, radio, television, electronic mail, electronic media, banner ads, and fiber optics. The entity may be a manufacturer or a retailer of the product, or a trade association whose members sell the product. The product may be identified by a trademark.

In another aspect, the invention relates to a method for inducing the consumption of calcium-containing products by a commercial entity having a financial interest in the sale of the products, wherein the entity distributes information to potential consumers of the products describing the benefits of reducing adipocytes by apoptosis attributable to the consumption of the products. In yet another aspect, the invention relates to a method for promoting the consumption of a calcium-containing product wherein said method comprises the public distribution of information describing the benefits of reducing adipocytes by apoptosis attributable to the consumption of the products.

The distribution of said information may be achieved by verbal communication, pamphlet distribution, print media, audio tapes, magnetic media, digital media, audiovisual media, billboards, advertising, newspapers, magazines, direct mailings, radio, television, electronic mail, braille, electronic media, banner ads, fiber optics, and laser light shows. The information may also pertain to a class of products to which said calcium-containing product belongs.

The subject invention also provides articles of manufacture useful in inducing adipocyte apoptosis in an individual intending or desiring to reduce the amount of adipocytes. In one embodiment, the invention may be an article of manufacture comprising a calcium-containing product and a description of an effect of consuming a calcium-containing dietary product, the described effect being inducing apoptosis in adipocytes. The description may be in the form of printed material and/or the product may be packaged and the description is part of the package. The description may directly accompany the product and/or is imprinted on the product. The printed materials may be in the form of pamphlets. The printed material may be embossed or imprinted on the product and may indicate the amounts of dietary calcium, recommended levels or serving amounts of dietary calcium or calcium-containing product intake necessary for inducing apoptosis of adipocytes, recommended BMI values, or recommended heights and weights for individuals. The description may also indicate the amounts of calcium contained within the product, and recommended levels of calcium intake for inducing apoptosis in adipocytes. In an exemplary embodiments, the products may be: cereal and the printed material is printed on the cereal box, milk and the printed material is printed on the milk container, cheese and the printed material is printed on the cheese package, yogurt and the printed material is printed on the yogurt container or animal food package and the printed material is printed on the animal food package.

The present invention also relates to methods of screening for or identifying an agent for inducing apoptosis in adipocytes, comprising: (a) treating adipocyte cells with a calcitrophic hormone and measuring a calcitrophic hormone activity, (b) treating said cells with a potential antagonist and measuring calcitrophic hormone activity, and (c) determining whether said calcitrophic hormone activity is inhibited by said potential antagonist. In one embodiment, the inhibited calcitrophic hormone activity may be one or more of lipogenesis, adiposity, triglyceride accumulation, elevated intracellular calcium concentration ([Ca²⁺]i), suppressed adipocyte uncoupling protein 2 (UCP2) expression, and/or stimulation of fatty acid syntliase (FAS) activity. The potential antagonist may be a vitamin D receptor antagonist or a vitamin D antagonist. In yet another aspect of the invention, the treating of cells with the calcitrophic hormone increases fatty acid synthase (FAS) activity and the increase may be prevented by pretreatment with the potential antagonist. In another exemplary aspect, the treating of cells with the calcitrophic hormone may inhibit lipolysis and the inhibition may be prevented by pretreatment with said potential antagonist.

In another exemplary embodiment, the method of screening or identifying an agent of calcitrophic hormone activity may comprise (a) treating human adipocyte cells with 1,25-dihydroxyvitamin D₃ (1,25-(OH)₂-D₃) and measuring calcitrophic hormone (1,25-(OH)₂-D₃) activity, (b) pre-treating said cells with a potential antagonist and measuring calcitrophic hormone (1,25-(OH)₂-D₃) activity, and (c) detecting inhibition of said calcitrophic hormone (1,25-(OH)₂-D₃) activity. In yet another aspect, the method may include (a) treating human adipocyte cells with 1,25-dihydroxyvitamin D (1,25-(OH)₂-D₃) and measuring calcitrophic hormone (1,25-(OH)₂-D₃) activity, (b) treating said cells with 1α,25-dihydroxylumisterol₃ (1α,25-(OH)₂-lumisterol₃) and measuring calcitrophic hormone (1,25-(OH)₂-D₃) activity, (c) pre-treating said cells with a potential antagonist and measuring calcitrophic hormone (1,25-(OH)₂-D₃) activity, and (d) detecting inhibition of said calcitrophic hormone (1,25-(OH)₂-D₃) activity by comparing the activity of step (c) with the activity of steps (a) and (b).

The order and numbering of the steps in the methods described herein are not meant to imply that the steps of any method described herein must be performed in the order in which the steps are listed or in the order in which the steps are numbered. The steps of any method disclosed herein can be performed in any order which results in a functional method. Furthermore, the method may be performed with fewer than all of the steps, e.g., with just one step.

Screening methods of the invention can be adapted to any of a variety of high throughput methodologies. High throughput assays are generally performed on a large number of samples, and at least some of the steps are performed automatically, e.g., robotically.

Another aspect of the invention is a kit, suitable for performing any of the methods of the invention. The components of the kit will vary according to which method is being performed. Reagents for performing suitable controls may also be included. Optionally, the kits comprise instructions for performing the method. Other optional elements of a kit of the invention include suitable buffers, media components, or the like; a computer or computer-readable medium for storing and/or evaluating assay results; logical instructions for practicing the methods described herein; logical instructions for analyzing and/or evaluating assay results as generated by the methods herein; containers; or packaging materials. The reagents of the kit may be in containers in which the reagents are stable. The kits and methods of the invention have many uses, which will be evident to the skilled worker. For example, they can be used in experiments to study the biological and chemical role calcitrophic hormone agonist and to identify calcitrophic hormone agonists that act as mimics, agonists or antagonists as described herein.

It should be understood that the examples and embodiments described herein are for illustrative purposes only and that various modifications or changes in light thereof will be suggested to persons skilled in the art and are to be included within the spirit and purview of this application and the appended claims.

### EXAMPLES

### Example 1

### Role of Uncoupling Protein 2 (UCP2) Expression and 1α,25-Dihydroxyvitamin D₃ in Modulating Adipocyte Apoptosis

Materials and Methods

Culture and Differentiation of 3T3-L1

3T3-L1 preadipocytes were incubated at a density of 8000 cells/cm2 (10 cm2 dish) and grown in Dulbecco's modified Eagle's medium (DMEM) containing 10% FBS and antibiotics (adipocyte medium) at 37° C. in 5% CO2 in air. Confluent preadipocytes were induced to differentiate with a standard differentiation medium consisting of DMEM-F10 (1:1, vol/vol) medium supplemented with 1% FBS, 1 μM dexamethasone, IBMX (0.5 mM), and antibiotics (1% penicillin-streptomycin). Preadipocytes were maintained in this differentiation medium for 3 days and subsequently cultured in adipocyte medium. Cultures were re-fed every 2-3 days to allow 90% cells to reach full differentiation before they were chemically treated. Chemicals were freshly diluted in adipocyte medium before treatment. Cells were washed with fresh adipocyte medium, re-fed with medium containing the different treatments, and incubated at 37° C. in 5% CO2 in air before analysis. Cell viability was measured via trypan blue exclusion.

UCP2 Transfection

UCP2 full-length cDNAs were amplified by real-time PCR(RT-PCR) using mRNAs isolated from mouse white adipose tissues. The PCR primers for this amplification are shown as follows: UCP2 forward, 5′-GCTAGCATGGTTGGTTTCAAG-3′, reverse, 5′-GCTAGCTCAGAAAGGTGAATC-3′. The PCR products were then subcloned into pcDNA4/His expression vectors. The linearized constructs were transfected into 3T3-L1 preadipocytes using lipofectamine plus standard protocol (Invitrogen, Carlsbad, Calif.). After 48 h of transfection, cells were split and cultured in selective medium containing 400 ug/ml zeocin for the selection of resistant colonies. Cells were fed with selective medium every 3 days until resistant colonies could be identified. These resistant foci were picked, expanded, tested for expression, and frozen for future experiments.

Determination of Mitochondrial Membrane Potential

Adipocytes were incubated with adipocyte medium for 4 h with or without 1α,25-(OH)₂-D₃. Mitochondrial membrane potential was analyzed fluorometrically with a lipophilic cationic dye JC-1 (5,5′,6,6′-tetrachloro-1,1′,3,3′-tetraethylbenzimidazol carbocyanine iodide) using a mitochondrial potential detection kit (Biocarta, San Diego, Calif.). Mitochondrial potential was determined as the ratio of red fluorescence (excitation 550 nm, emission 600 nm) and green fluorescence (excitation 485 nm, emission 535 nm) using a fluorescence microplate reader.

ATP Measurement

Adipocytes were incubated with adipocyte medium for 4 h with or without 1α,25-(OH)₂-D₃. ATP levels were measured using ENLITEN Total ATP Rapid Biocontamination Detection Kit (Promega, Madison, Mich.) according to the manufacturer's instructions. Cellular ATP was extracted as described previously and measured using a microplate luminometer (Labsystem, Helsinki, Finland). The integration time of the luminometer was set at 1 s with normal gain. Measurement of cytosolic Ca²⁺ ([Ca²⁺]c) and mitochondrial calcium([Ca²⁺]m)

[Ca²⁺]c in adipocytes was measured using a fura-2 dual-wavelength fluorescence imaging system. Cells were plated in 35-mm dishes (P35G-0-14-C, MatTek). Before [Ca²⁺]c measurement, cells were put in serum-free medium overnight and rinsed with HEPES balanced salt solution (HBSS) containing the following components (in mM): 138 NaCl, 1.8 CaCl₂, 0.8 MgSO₄, 0.9 NaH₂PO₄, 4 NaHCO₃, 5 glucose, 6 glutamine, 20 HEPES, and 1% bovine serum albumin. Cells were loaded with fura-2 acetoxymethyl ester (fura-2 AM) (10 μM) in the same buffer for 2 h at 37° C. in a dark incubator with 5% CO₂. To remove extracellular dye, the cells were rinsed with HBSS three times and then postincubated at room temperature for an additional 1 h for complete hydrolysis of cytoplasmic fura-2 AM. The dishes with dye-loaded cells were mounted on the stage of Nikon TMS-F fluorescence inverted microscope with a Cohu model 4915 charge-coupled device (CCD) camera. Fluorescent images were captured alternatively at excitation wavelengths of 340 and 380 nm with an emission wavelength of 520 nm. After establishment of a stable baseline, the responses to 1α,25-(OH)₂-D₃ were determined. [Ca²⁺]c was calculated using a ratio equation as described previously. Each analysis evaluated responses of five representative whole cells. Images were analyzed with InCyt Im2 version 4.62 imaging software (Intracellular Imaging, Cincinnati, Ohio). Images were calibrated using a fura-2 calcium imaging calibration kit (Molecular Probes, Eugene, Oreg.) to create a calibration curve in solution, and cellular calibration was accomplished using digitonin (25 μM) and pH 8.7 Tris-EGTA (100 mM) to measure maximal and minimal [Ca²⁺]c levels, respectively.

Rhod-2/AM was used for qualitative measurement of [Ca²⁺]m. Cells were incubated with the Ca²⁺-sensitive fluorescent indicator rhod-2 AM (1-1.5 μM) for at least 1 h at 37° C. in 5% CO₂ in humidified air. Because rhod-2 AM consists of a cationic rhodamine molecule, it accumulates preferentially inside the mitochondria due to their negative membrane potential. After loading, the cells were kept in rhod-2 AM-free standard solution for at least 1 h to allow conversion of the dye to its Ca²⁺-sensitive, free acid form. Images of [Ca²⁺]m were acquired at the wavelength of 552 nm excitation and 590 nm emission using the intracellular imaging system described above. Because rhod-2 is not a ratiometric dye, its fluorescence intensity was not calibrated to obtain absolute values of [Ca²⁺]m. Instead, only relative values were recorded as fluorescence signal (F) relative to the control value (F0). After baseline calcium concentration was measured, the 1α,25-(OH)₂-D₃ was immediately applied to the cells and the calcium concentration was measured again.

Total RNA Extraction

A total cellular RNA isolation kit (Ambion, Austin, Tex.) was used to extract total RNA from 3T3-L1 cells according to the manufacturer's instructions.

Quantitative Real-Time PCR(RT-PCR)

Adipocyte caspase-1, caspase-3, Bcl-2, and Bax mRNA were quantitatively measured using a Smart Cycler Real Time PCR System (Cepheid, Sunnyvale, Calif.) with a TaqMan 1000 Core Reagent Kit (Applied Biosystems, Branchburg, N.J.). The primers and probe sets were ordered from Applied Biosystems TaqMan Assays-on-Demand Gene Expression primers and probe set collection according to the manufacturer's instructions. Pooled 3T3-L1 adipocyte total RNA was serial-diluted in the range of 1.5625-25 ng and used to establish a standard curve; total RNAs for unknown samples were also diluted in this range. Reactions of quantitative RT-PCR for standards and unknown samples were Cepheid, Sunnyvale, Calif.) and TaqMan Real Time PCR Core Kit. The mRNA quantitation for each sample was further normalized using the corresponding 18 s quantitation, with a forward primer, 5′-AGTCCCTGCCCTTTGTACACA-3′, and a reverse primer, 5′-GATCCGAGGGCCTCACTAAAC-3′.

Animals and Diets

This study was divided into three phases. The first phase was designed to induce weight gain and fat accretion, the second phase studied acceleration of weight and fat loss on the obese animals induced by phase one, and the third phase determined attenuation of gaining back weight and fat. The study was conducted in the aP2 transgenic mice used in the studies described previously. These mice exhibit a normal pattern of leptin gene expression and activity as well as a pattern of agouti gene expression similar to that found in humans. Therefore, these mice are useful models for diet-induced obesity in a genetically susceptible human population in that they are not obese on a standard AlN-93 G diet, but develop mild to moderate obesity when fed high-sucrose and/or high-fat diets.

Phase I

Six-wk-old aP2 transgenic mice were studied in a six-wk obesity induction period on a basal low-calcium/high-sucrose/high-fat diet. Sixty aP2 transgenic mice from a colony were placed at 6 wk of age on a modified AlN 93 G diet with suboptimal calcium (0.4%), sucrose as the sole carbohydrate source and providing 64% of energy, and fat increased to 25% of energy with lard. Animals were studied for 6 wk, during which time food intake and spillage were measured daily, and body weight and food consumption were assessed weekly. At the end of phase I, five representative animals were killed to collect fat pads as described under Phase III.

Phase II

At the end of the phase I, the 55 animals remaining were put on a high-calcium diet, but the energy intake was limited to 70% of that found in the ad libitum fed during phase I. The high-calcium diet used in this phase was made of the basal diet with the addition of sufficient calcium to bring the calcium content of the diet to 1.3%. Animals were maintained on this diet for 6 wk, during which time food intake and spillage were measured daily and body weight and food consumption were assessed weekly. At the end of phase II, five animals were killed to collect fat pads, as described under Phase III.

Phase III

At the end of phase II, the 50 animals remaining were further randomized into five groups, as following: 1) basal-re-fed group was put on high-sucrose/high-fat/low-calcium basal diet ad libitum; 2) high-calcium cereal-re-fed group was put on high-calcium (1.3%) cereal diet ad libitum; 3) high-calcium cereal plus dry-milk group was put on high-calcium cereal plus nonfat milk diet ad libitum, in which 1.2% calcium content came from fortified cereal and 0.1% calcium content came from nonfat milk; 4) yogurt-re-fed group was put on high-calcium (1.3%) yogurt diet ad libitum; and 5) cereal control-re-fed group was put on low-calcium (0.4%) cereal diet ad libitum. Ten animals per group were studied for 6 wk, during which food intake and spillage were measured daily and body weight and food consumption were assessed weekly. At the conclusion of the study, all mice were killed under isofluorane anesthesia, and fat pads were immediately excised, weighed, and snap-frozen in liquid nitrogen for subsequential assessment of gene expression.

Statistical Analysis

All data are expressed as mean±se. Data were evaluated for statistical significance by one-way ANOVA, and significantly different group means were then separated by the least significant difference test by using SPSS (Chicago, Ill.).

Results

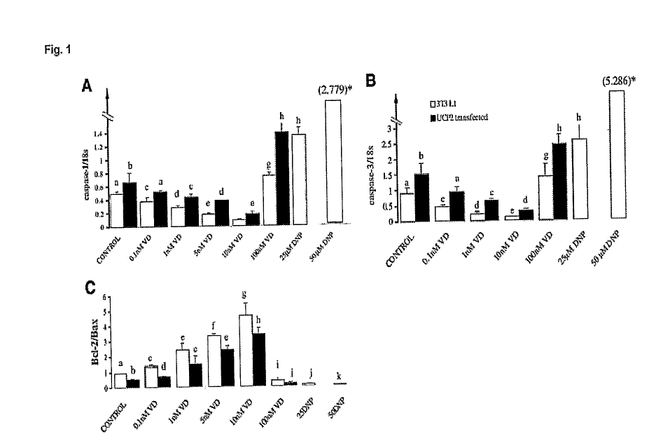

All apoptosis signaling pathways converge on a common pathway of cell destruction that is activated by a family of cysteine proteases (caspases) that cleave protein at aspartate residues. Accordingly, the effect of 1α,25-(OH)₂-D₃ and DNP on caspase-1 and caspase-3 expression in differentiated 3T3-L1 (control) and UCP2-transfected 3T3-L1 cells were studied. FIGS. 1A and 1B demonstrate that physiological low doses of 1α,25-(OH)₂-D₃ (0.1 nM, 1 nM, 5 nM, and 10 nM) inhibited caspase-1 and caspase-3 expression, respectively (P<0.05), indicating reduced apoptosis in both control and UCP2-transfected 3T3-L1 cells. In contrast, a high dose of 1α,25-(OH)₂-D₃ (100 nM) stimulated both caspase-1 and caspase-3 expression in both control and UCP2-transfected 3T3-L1 cells (P<0.01), indicating a pro-apoptotic effect. Although 1α,25-(OH)₂-D₃ exerted similar effects on caspase-1 and -3 expression in 3T3-L1 and UCP2-transfected 3T3-L1 cells, the basal expression levels of these two caspases were significantly higher in the DNP-treated and UCP2-transfected cells compared with control cells (P<0.05), suggesting that mitochondrial uncoupling stimulates apoptosis in cultured 3T3-L1 cells.

Both Bcl-2 and Bax are apoptotic proteins belonging to the Bcl-2 family. Due to their completely different roles in the apoptotic signaling pathway, the ratio of protective Bcl-2 to apoptotic Bax is widely used to determine the susceptibility to apoptosis by regulating mitochondrial function following an apoptotic stimuli. FIG. 1C shows the effect of 1α,25-(OH)₂-D₃ on Bcl-2/Bax expression ratio. Lower levels of 1α,25-(OH)₂-D₃ were found to induce a dose-dependent increase in Bcl-2/Bax ratio (P<0.01), indicating a protective role of lower level of 1α,25-(OH)₂-D₃ against apoptosis. In contrast, the high dose of 1α,25-(OH)₂-D₃ resulted in a significant decrease in the Bcl-2/Bax ratio in control and UCP2-transfected 3T3-L1 cells (P<0.01). In addition, the DNP-treated and UCP2-transfected cells showed significantly lower ratio of Bcl-2/Bax than nontransfected 3T3-L1 cells (P<0.02), indicating that mitochondrial uncoupling induced a decrease in protection from apoptotic death.

Because a physiological low dose of 1α,25-(OH)₂-D₃ appears to inhibit adipocyte apoptosis, suppression of 1α,25-(OH)₂-D₃ with a high-calcium diet may stimulate apoptosis. Accordingly, a comparison was conducted on caspase-1 and caspase-3 expression as well as Bcl-2/Bax expression ratio in aP2 transgenic mice fed diets with different calcium content after energy restriction. FIG. 2A-C show that significant higher increases in caspase-1 and caspase-3 expression (P<0.001) and decreases in Bcl-2/Bax ratio in mice fed high-calcium diets (Ca 1.3%) than in mice fed low-calcium diets (Ca 0.4%) (P<0.01). These data suggest that suppression 1α,25-(OH)₂-D₃ physiologically in vivo may stimulate adipocytes apoptosis.

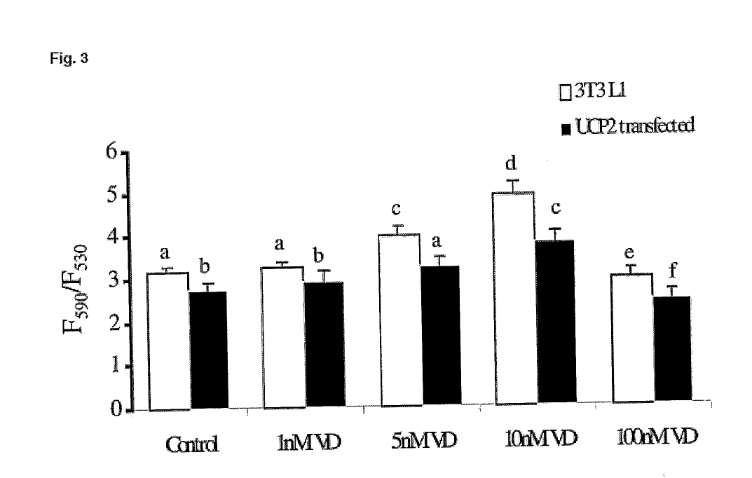

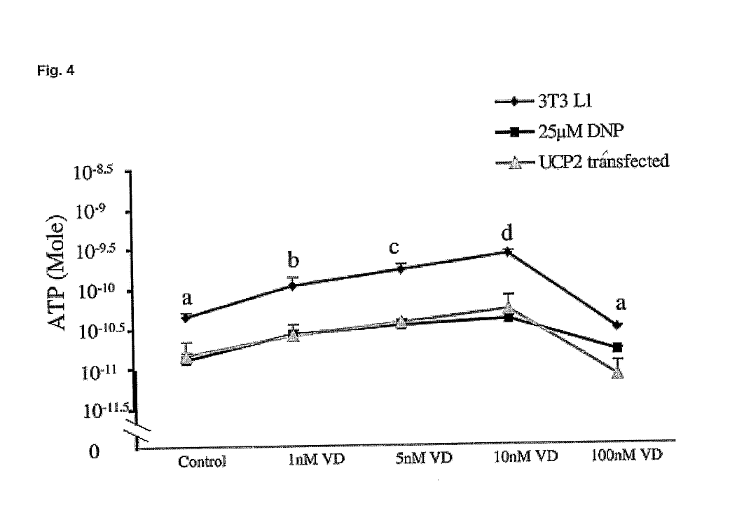

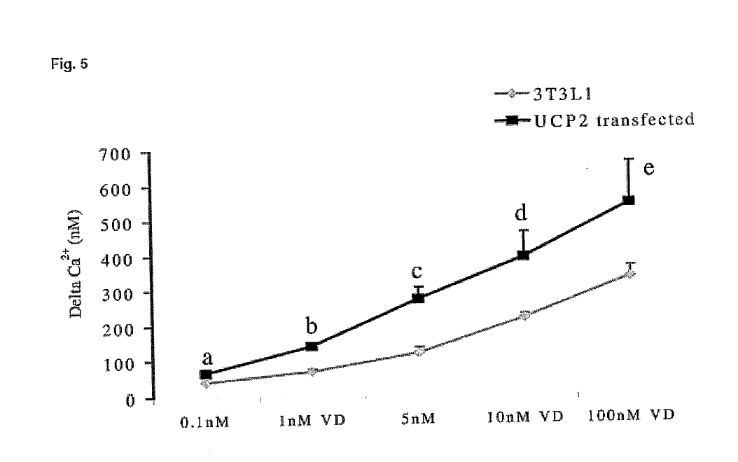

FIG. 3 shows the effect of 1α,25-(OH)₂-D₃ on mitochondrial potential in 3T3-L1 and UCP2 stably transfected 3T3-L1 cells. Lower doses of 1α,25-(OH)₂-D₃ were found to significantly increase mitochondrial potential in all three groups of cells, whereas the high dose of 1α,25-(OH)₂-D₃ caused mitochondrial potential collapse. Consistent with these data, ATP production was markedly increased in response to low-dose 1α,25-(OH)₂-D₃ treatment (P<0.01) (FIG. 4), and the high dose of 1α,25-(OH)₂-D₃ inhibited ATP production (P<0.05). These effects are consistent with the inventors previous observation that the low dose of 1α,25-(OH)₂-D₃ inhibited UCP2 expression. Thus, by inhibiting UCP2 expression, the low dose of 1α,25-(OH)₂-D₃ restored mitochondrial potential and increased ATP production. 1α,25-(OH)₂-D₃ also stimulated Ca²⁺ influx in both control and UCP2-transfected cells. FIG. 5 demonstrates a dose-dependent increase in cytosolic calcium levels in response to 1α,25-(OH)₂-D₃. Notably, the high dose of 1α,25-(OH)₂-D₃ induced a 10-fold higher increase in calcium influx than the lower doses.

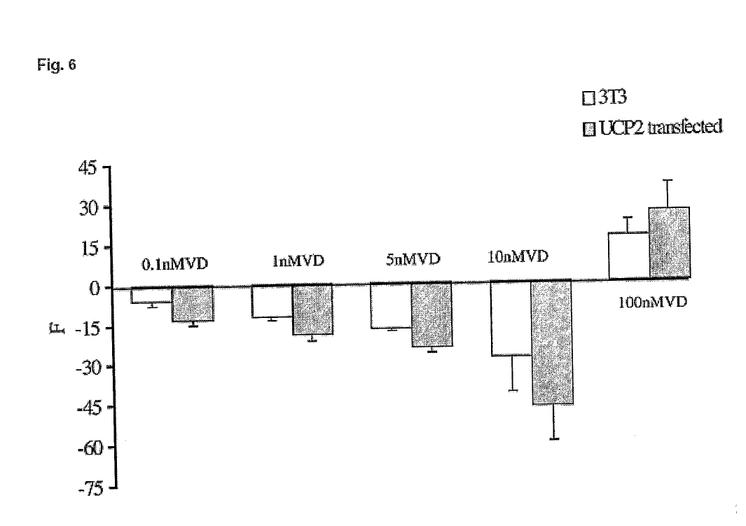

FIG. 6 shows the effect of 1α,25-(OH)₂-D₃ on [Ca²⁺]m levels in control and UCP2-transfected cells. Low doses of 1α,25-(OH)₂-D₃ decreased mitochondrial calcium in both types of cells, and the high dose of 1α,25-(OH)₂-D₃ significantly stimulated [Ca²⁺]m. Because mitochondrial calcium overload might trigger apoptosis by inducing mitochondrial collapse and cytochrome c release, the pro-apoptotic effect of the high dose of 1α,25-(OH)₂-D₃ is most likely a result of the stimulation of [Ca²⁺]m. In contrast, the decreases in [Ca²⁺]m observed in response to lower doses of 1α,25-(OH)₂-D₃ indicate that, in addition to the inhibitory effect on UCP2, reducing the mitochondrial calcium load may contribute to the anti-apoptotic effect of low doses of 1α,25-(OH)₂-D₃.

Discussion

Example 1 demonstrates that lower (physiological) doses of 1α,25-(OH)₂-D₃ inhibit apoptosis in differentiated 3T3-L1 adipocytes, and the suppression of 1α,25-(OH)₂-D₃ in vivo by increasing dietary calcium stimulates adipocyte apoptosis during refeeding following energy restriction in aP2 transgenic mice, suggesting that the stimulation of adipocyte apoptosis contributes to reduced adipose tissue mass after administration of high-calcium diets.

Key features of apoptosis involve the proteolytic caspases as well as apoptotic proteins Bcl-2 and Bax. Activation of Bax induces apoptosis by disturbing mitochondrial electron transport chain, counters the death repressor activity of Bcl-2, and promotes the release of cytochrome c into the cytoplasm. This, in turn, activates caspases that initiate execution of apoptotic death. Bcl-2, which is localized to mitochondria, inhibits Bax-induced apoptosis, and, consequently, the Bcl-2/Bax ratio can be used to determine the susceptibility to apoptosis. Example 1 shows that lower doses of 1α,25-(OH)₂-D₃ (0.1 nm, 1 nm, 5 nm, and 10 nm) dose-dependently inhibit caspase-1 and caspase-3 gene expression and increase the Bcl-2/Bax expression ratio in control and UCP2-transfected 3T3-L1 adipocytes, indicating an inhibition of apoptosis. UCP2-transfected 3T3-L1 adipocytes were found to have a higher basal level of caspase-1 and caspase-3 expression but a lower Bcl-2/Bax ratio than nontransfected 3T3-L1 cells. This is consistent with the observation that UCP2 stimulates apoptosis by inducing mitochondrial potential collapse and inhibiting ATP production.

1α,25-(OH)₂-D₃ inhibits UCP2 expression in human adipocytes and 1α,25-(OH)₂-D₃ was found to functionally inhibit UCP2 action by increasing mitochondrial potential and ATP production in both nontransfected and UCP2-transfected 3T3-L1 cells. Thus, the suppression of apoptosis induced by 1α,25-(OH)₂-D₃ is, in part, mediated by inhibiting UCP2 expression and activity. Suppression of 1α,25-(OH)₂-D₃ secondary to consumption of high-calcium diets may stimulate adipocyte apoptosis in vivo. This was evaluated in aP2 transgenic mice undergoing re-feeding following energy restriction and found a significant increase in white adipose tissue apoptosis in mice re-fed high-calcium diets (1.3%) compared with low-calcium diets (0.4% Ca). These results further confirm that suppression of 1α,25-(OH)₂-D₃ stimulates apoptosis in white adipose tissue and suggest that this effect also contributes to the anti-obesity effect of dietary calcium.

In contrast, very high doses of 1α,25-(OH)₂-D₃, which are three- to fourfold higher than physiological levels, exert the opposite effect. A high dose of 1α,25-(OH)₂-D₃ (100 nM) actually stimulated caspase-1 and caspase-3 expression and inhibited Bcl-2/Bax ratio, a complete reversal of the effect of lower doses of 1α,25-(OH)₂-D₃ on apoptosis. Although lower doses of 1α,25-(OH)₂-D₃ caused moderate increases in cytosolic [Ca²⁺] levels in adipocytes, the high dose of 1α, 25-(OH)₂-D₃ induced an extreme elevation, and excessive intracellular calcium has been reported to be a pro-apoptotic factor.

In Example 1, mitochondrial Ca²⁺ concentration were monitored and it was found that low does of 1α,25-(OH)₂-D₃ decreased mitochondrial Ca²⁺ levels in a dose-dependent manner, whereas a high dose of 1α,25-(OH)₂-D₃ markedly stimulated the elevation of [Ca²⁺]m, indicating that the increase in [Ca²⁺]m is associated with the induction of apoptosis by a high dose of 1α,25-(OH)₂-D₃. Mitochondrial uncoupling stimulated the [Ca²⁺]c level, which may in turn induce an increase in [Ca²⁺]m to maintain cytoplasmic calcium homeostasis. Low doses of 1α,25-(OH)₂-D₃ induce dose-dependent depletion [Ca²⁺]m by inhibiting UCP2, thereby protecting adipocytes from apoptotic death. High doses of 1α,25-(OH)₂-D₃, on the other hand, increase [Ca²⁺]c to an extreme level, which may increase both [Ca²⁺]m and [Ca²⁺]er. Because ER can open their Ca²⁺ release channel in response to elevations in [Ca²⁺]c and contribute to Ca²⁺-induced Ca²⁺ release (CICR), this may further increase [Ca²⁺]m, and calcium overload in mitochondria in turn triggers apoptosis.

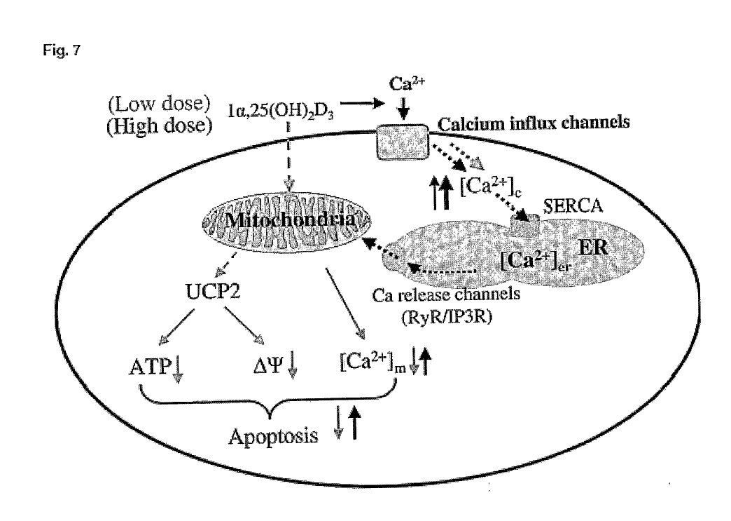

In summary, Example 1 shows the dual effects of 1α,25-(OH)₂-D₃ on apoptotic death. These are summarized in FIG. 7 as follows: by inhibiting UCP2 and decreasing mitochondrial calcium, lower (physiological) doses of 1α,25-(OH)₂-D₃ inhibit apoptosis in adipocytes, while suppression of 1α,25-(OH)₂-D₃ using high-calcium diets stimulates adipose apoptosis in mice. In contrast, a high dose of 1α,25-(OH)₂-D₃ stimulates mitochondrial calcium overload and apoptosis in adipocytes. These results indicate that dietary calcium not only regulates adipocyte size by increasing lipid accumulation, but also modulates adipocyte number by stimulating apoptotic death.

### Example 2

### Mechanisms of Dual Effects of 1α,25-Dihydroxyvitamin D₃ on Adipocyte

Apoptosis

Materials and Methods

Culture and Differentiation of 3T3-L1 Adipocytes

3T3-L1 preadipocytes were incubated at a density of 8000 cells/cm2 (10 cm2 dish) and grown in Dulbecco's modified Eagle's medium (DMEM) containing 10% FBS and antibiotics at 37° C. in 5% CO₂. Confluent preadipocytes were induced to differentiate with a standard differentiation medium consisting of DMEM-F 10 (1:1, vol/vol) medium supplemented with 1% FBS, 1 μM dexamethasone, IBMX (0.5 mM) and antibiotics (1% penicillin/streptomycin). Preadipocytes were maintained in this differentiation medium for 3 days and subsequently cultured in adipocyte medium. Cultures were re-fed every 2-3 days to allow 90% cells to reach fully differentiation before conducting chemical treatment. Chemicals were freshly diluted in adipocyte medium before treatment. Cells were washed with fresh adipocyte medium, re-fed with medium containing the different treatments, and incubated at 37° C. in 5% CO₂ in air before analysis. Cell viability was measured via trypan blue exclusion.

UCP2 Transfection

UCP2 full-length cDNAs were amplified by RT-PCR using mRNAs isolated from mouse brown and white adipose tissues, respectively. The PCR primers for this amplification are shown as follows: UCP2 forward, 5′-GCTAGCATGGTTGGTTTCAAG-3′, reverse, 5′-GCTAGCTCAGAAAGGTGAATC-3′. The PCR products were then subcloned into pcDNA4/His expression vectors. The linearized constructs were transfected into 3T3-L1 preadipocytes using lipofectamine plus standard protocol (Invitrogen, Carlsbad, Calif.). After 48 hrs of transfection, cells were split and cultured in selective medium containing 400 ug/ml zeocin for the selection of resistant colonies. Cells were fed with selective medium every 3 days until resistant colonies were identified. These resistant foci were picked, expanded, tested for expression, and frozen for future experiments.

siRNA Preparation and Transfections.

Duplex siRNA corresponding to UCP2 gene was designed and chemically synthesized by Invitrogen. The following gene-specific sequences were used successfully: Si-UCP2 sense 5′-GCCUCUACGACUCUGUCAA-3′ and antisense 5′-UUGACAGAGUCGAGGC-3′. Transfection of siRNA was performed with Oligofectamine™ reagent (Invitrogen Life Technologies, CA) in 6-well plates. Briefly, Oligofectamine diluted in DMEM was applied to the duplex siRNA mixture, and the formulation was continued for 20 min to allow the Oligofectamine-oligonucleotide complex to form. Per well, 800 μl of plating medium and 10 ul of 20 μM duplex siRNA duplex siRNA formulated with 4 μl of Oligofectamine were applied in a final volume of 1 ml. Cells were then incubated at 370 degrees Celsius in a CO₂ incubator for 4 hrs. 500 ul of DMEM medium containing 3× serum was added to transfection mixture after transfection, and cells were incubated for 48 h˜72 h at 370 degrees Celsius in a CO₂ incubator.

Western Blot Analysis

Adipocytes were harvested and sonicated in a homogenization buffer containing 50 mM Tris-HCl (PH 7.4), 250 mM sucrose, 1 mM EDTA, 1 mM dithiothreitol, 1% (v/v) Triton X, 10% protease cocktail (Sigma). Cell homogenates were incubated on ice for 1 h to solublize all proteins, and the insoluble protein was removed by centrifugation at 12,500 g at 40 degrees Celsius for 15 min. Homogenate intrafranatant protein from equal numbers of cells (determined via protein quantitation using Bradford method as described previously) was boiled in Laemmli sample buffer and subject to 10% SDS-polyacrylamide gel electrophoresis (PAGE). Proteins on the gels were transferred to Hybond ECL nitrocellulose membrane (Amersham Pharmacia Biotech, Piscataway, N.J.). The transferred membranes with proteins were blocked, washed, incubated with 1:1000 dilution of anti-UCP2 polyclonal antibody or 1:500 dilution of anti-actin polyclonal antibody (Santa Cruz Biotechnology, CA) followed by peroxidase-conjugated secondary antibody (Santa Cruz Biotechnology, CA). Visualization was detected with chemiluminescence reagent, using the Western Blotting Luminal Reagent (Santa Cruz Biotechnology, CA).

Determination of Mitochondrial Membrane Potential

Adipocytes were incubated with adipocyte medium for 4 h with or without GDP, BK 8644, GDP plus BK8644. Mitochondrial membrane potential was analyzed fluorometrically with a lipophilic cationic dye JC-1 (5,5′,6,6′-tetrachloro-1,1′,3,3′-tetraethylbenzimidazol carbocyanine iodide) using a mitochondrial potential detection kit (Biocarta, San Diego, Calif.). Mitochondrial potential was determined as the ratio of red fluorescence (excitation 550 nm, emission 600 nm) and green fluorescence (excitation 485 nm, emission 535 nm) using a fluorescence microplate reader.

Measurement of cytosolic Ca²⁺ ([Ca²⁺]c) and mitochondrial calcium([Ca²⁺]m).

Adipocytes were incubated with adipocyte medium for 4 h with or without GDP, BK 8644 before assay. [Ca²⁺]c in adipocytes was measured using a fura-2 dual-wavelength fluorescence imaging system. Cells were plated in 35-mm dishes (P35G-0-14-C, MatTek). Prior to [Ca²⁺]c measurement, cells were put in serum-free medium overnight and rinsed with HEPES balanced salt solution (HBSS) containing the following components (in mM): 138 NaCl, 1.8 CaCl₂, 0.8 MgSO₄, 0.9 NaH₂PO₄, 4 NaHCO₃, 5 glucose, 6 glutamine, 20 HEPES, and 1% bovine serum albumin. Cells were loaded with fara-2 acetoxymethyl ester (fura-2 μM) (10 μM) in the same buffer for 2 h at 37° C. in a dark incubator with 5% CO₂. To remove extracellular dye, cells were rinsed with HBSS three times and then post-incubated at room temperature for an additional 1 h for complete hydrolysis of cytoplasmic fura-2 AM. The dishes with dye-loaded cells were mounted on the stage of Nikon TMS-F fluorescence inverted microscope with a Cohu model 4915 charge-coupled device (CCD) camera. Fluorescent images were captured alternatively at excitation wavelengths of 340 and 380 nm with an emission wavelength of 520 nm. After establishment of a stable baseline, the responses to 1α,25-(OH)₂-D₃ was determined. [Ca²⁺]c was calculated using a ratio equation as described previously. Each analysis evaluated responses of 5 representative whole cells. Images were analyzed with InCyt Im2 version 4.62 imaging software (Intracellular Imaging, Cincinnati, Ohio). Images were calibrated using a fura-2 calcium imaging calibration kit (Molecular Probes, Eugene, Oreg.) to create a calibration curve in solution, and cellular calibration was accomplished using digitonin (25 μM) and pH 8.7 Tris-EGTA (100 mM) to measure maximal and minimal [Ca²⁺]c levels respectively.

Rhod-2/AM was used for qualitative measurement of [Ca²⁺]m. Cells were incubated with the Ca²⁺ sensitive fluorescent indicator rhod-2 AM (1-1·5 μM), for at least 1 h at 370 degrees Celsius in 5% CO₂ in humidified air. Since rhod-2 AM consists of a cationic rhodamine molecule, it accumulates preferentially inside the mitochondria due to their negative membrane potential. After loading, the cells were kept in rhod-2 AM-free standard solution for at least 1 h to allow conversion of the dye to its Ca²⁺-sensitive, free acid form. Images of [Ca²⁺]m were acquired at the wavelength of 552 nm excitation and 590 nm emission using the intracellular imaging system described above. Since rhod-2 is not a ratiometric dye, its fluorescence intensity was not calibrated to obtain absolute values of [Ca²⁺]m. Instead, only relative values were recorded as fluorescence signal (F) relative to the control value (F0).

Total RNA Extraction.

Adipocytes were incubated with adipocyte medium for 24 h with or without GDP, BK 8644, GDP plus BK8644 before total RNA extraction. A total cellular RNA isolation kit (Ambion, Austin, Tex.) was used to extract total RNA from 3T3-L1 cells according to manufacturer's instruction.

Quantitative Real Time PCR

Adipocyte caspase-3 was quantitatively measured using a Smart Cycler Real Time PCR System (Cepheid, Sunnyvale, Calif.) with a TaqMan 1000 Core Reagent Kit (Applied Biosystems, Branchburg, N.J.). The primers and probe sets were designed and synthesised by Applied Biosystems TaqMan® Assays-on-Demand™ Gene Expression primes and probe set collection according to manufacture's instruction. Pooled 3T3-L1 adipocyte total RNA was serial-diluted in the range of 1.5625-25 ng and used to establish a standard curve; total RNAs for unknown samples were also diluted in this range. Reactions of quantitative RT-PCR for standards and unknown samples were also performed according to the instructions of Smart Cycler System (Cepheid, Sunnyvale, Calif.) and TaqMan Real Time PCR Core Kit (Applied Biosystems, Branchburg, N.J.). The mRNA quantitation for each sample was further normalized using the corresponding 18s quantitation, with a forward primer: 5′-AGTCCCTGCCCTTTGTACACA-3′ and a reverse primer: 5′-GATCCGAGGGCCTCACTAAAC-3′.

Statistical Analysis

All data are expressed as mean±SE. Data were evaluated for statistical significance by one-way analysis of variance (ANOVA), and significantly different group means were then separated by the least significant difference test by using SPSS (SPSS Inc, Chicago, Ill.).

Results

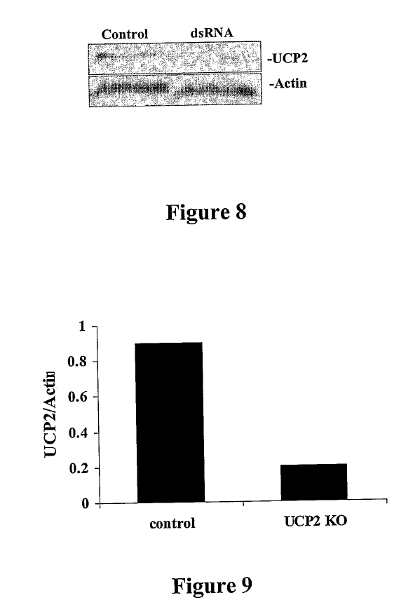

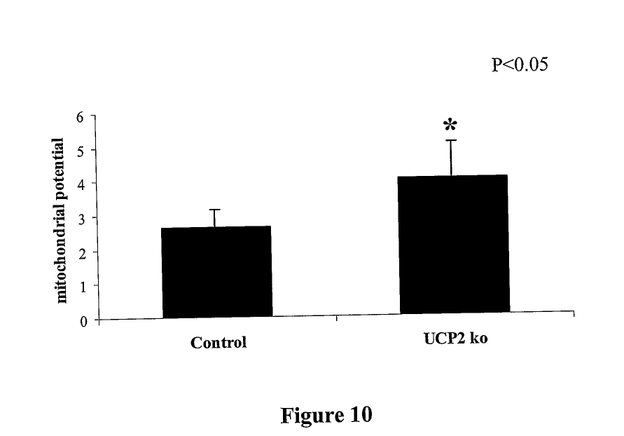

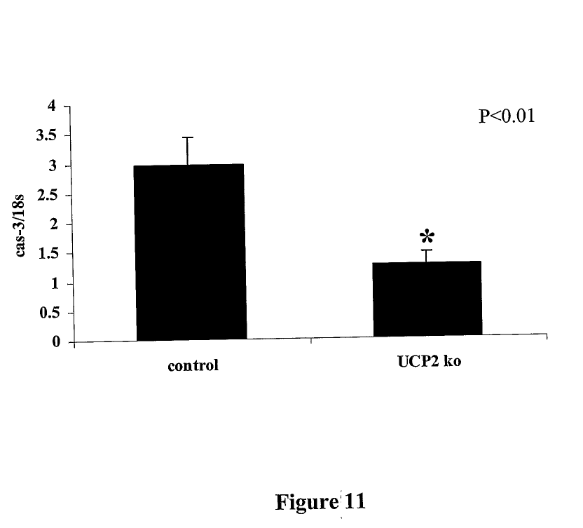

The increase of mitochondrial uncoupling by either chemical uncouplers or UCP2 overexpression in 3T3-L1 cells stimulates apoptosis, with significant increases in caspase-1 and caspase-3 expression as well as decrease in Bcl-2/Bax expression ratio. Consistent with this observation, it was found that suppression of UCP2 by siRNA transfection in 3T3-L1 cells caused a 70% decrease in UCP2 expression and a 52% increase in mitochondrial potential (p<0.05) (FIGS. 8, 9 and 10), indicating that suppression of UCP2 increases adipocyte mitochondrial potential. Inhibition of UCP2 expression in transfected cells also induced a 58% decrease in caspase 3 expression (p<0.01) (FIG. 11). These data suggest that UCP2 plays a direct role in stimulation of adipocyte apoptosis. Accordingly, suppression of UCP2 expression decreases apoptotic stress.

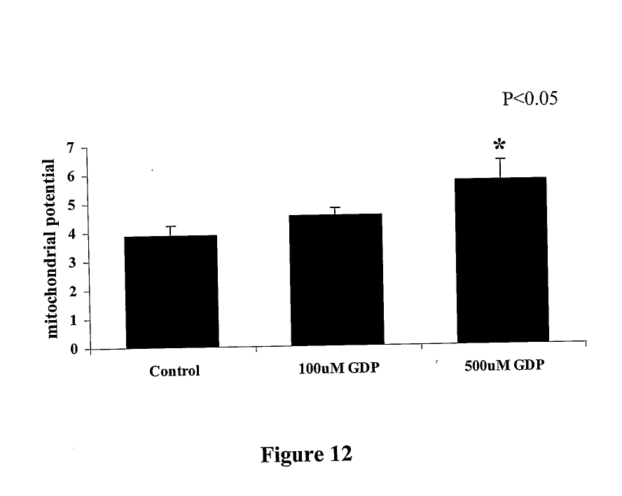

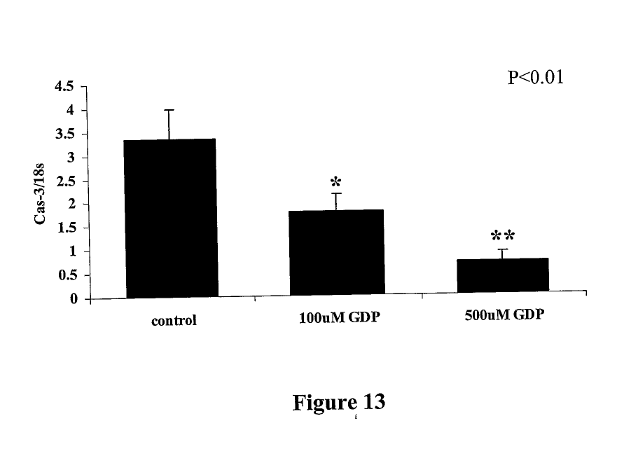

Although overexpression of UCP2 favors adipocyte apoptosis while suppression of UCP2 decreases apoptotic stress, it is not clear whether other UCP isoforms may also be regulated correspondingly during UCP2 manipulation and thereby affect apoptosis. Accordingly, GDP was used as a general mitochondrial uncoupling inhibitor to investigate the effect of mitochondrial uncoupling inhibition on apoptosis. GDP at 100 μM and 500 μM increased mitochondrial potential by 27% and 48% (p<0.05) respectively (FIG. 12), confirming an inhibitory effect of GDP on mitochondrial uncoupling. FIG. 13 shows that GDP treatments inhibited apoptosis in 3T3-L1 cells, with 47%-81% decreases in caspase-3 expression (p<0.01), indicating that general inhibition of mitochondrial uncoupling suppresses adipocyte apoptosis.

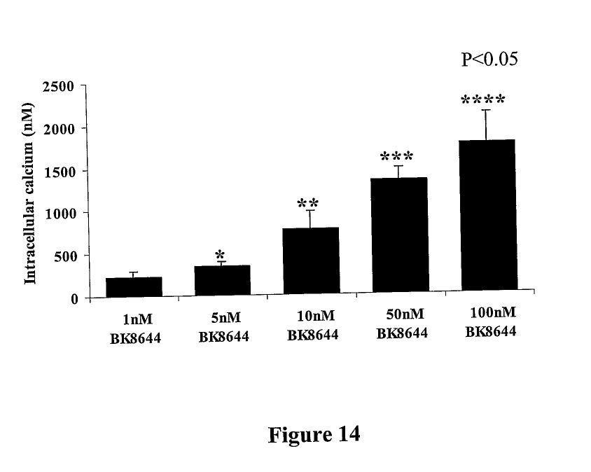

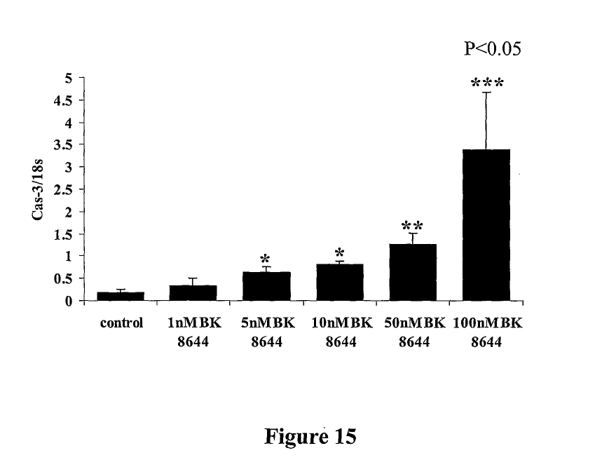

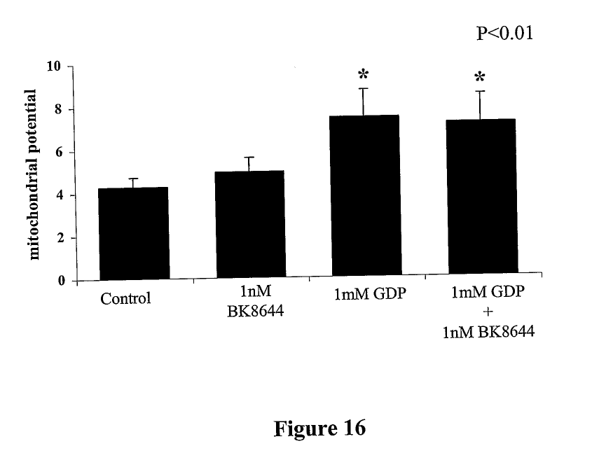

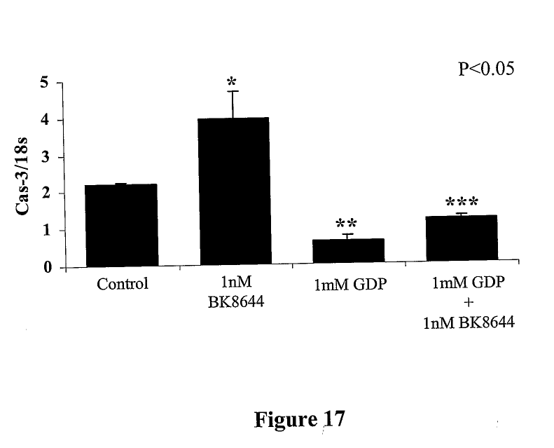

Bay K 8644 (BK 8644) is a Ca²⁺ ionophore which stimulated adipocyte Ca²⁺ influx in a dose-dependent manner (FIG. 14). This stimulation of BK8644 (1 nM-100 nM) on [Ca²⁺]i is of comparable magnitude to the effect of 1α,25-(OH)₂-D₃. However, BK8644 exerted no effect on mitochondrial potential or UCP2 expression (FIG. 16). This result is consistent with previous observations that 1α,25-(OH)₂-D₃ regulates UCP2 expression via a calcium-independent mechanism. Notably, Bay K 8644 stimulated caspase-3 expression dose-dependently (FIG. 15), indicating that increases in [Ca²⁺]i influx independently stimulate adipocyte apoptosis. Consistent with the observation that mitochondrial uncoupling increases cytosolic calcium level, inhibition of mitochondrial uncoupling with GDP decreased Ca²⁺]i level significantly (data not shown). Combining 1 mM GDP with 1 nM BK 8644 exerted no effect on [Ca²⁺]i but induced similar magnitude of increases in mitochondrial potential as observed in GDP alone (FIG. 16), indicating that the regulation of mitochondrial potential is calcium independent. However, addition of BK 8644 caused a significant increase in caspase-3 expression (FIG. 17), suggesting that [Ca²⁺]i stimulates apoptosis independent of mitochondrial potential.

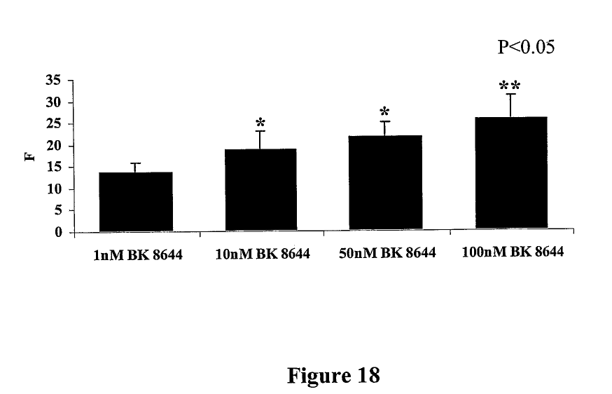

Stimulation of calcium influx by BK 8644 increased mitochondrial calcium (FIG. 18) (p<0.05). Although 1α,25-(OH)₂-D₃ induced similar effects on [Ca²⁺]i increase as BK8644, the opposite effect on mitochondrial calcium was observed with low dose of 1α,25-(OH)₂-D₃ (0.1 nM to 10 nM) pretreated cells, with significant decreases in mitochondrial calcium. The high dose of 1α,25-(OH)₂-D₃, however, caused similar increases in mitochondrial calcium as observed with BK 8644.

Discussion

The effects of the calcium channel ionophore BK 8644, a mitochondrial uncoupling inhibitor (GDP) and UCP2 silencing using siRNA transfection on apoptosis were investigated. BK 8644, which exerted no effect on mitochondrial uncoupling, caused a dose dependent increase in [Ca²⁺]i as well as mitochondrial calcium storage, and stimulated caspase-3 expression, indicating that increases in [Ca²⁺]i influx, along with mitochondrial calcium load, stimulate apoptosis. Inhibition of mitochondrial uncoupling with GDP decreased [Ca²⁺]i and mitochondrial potential and thereby inhibited apoptosis. Consistent with the observation that the mitochondrial uncoupler 2,4-dinitrophenol (DNP) stimulates apoptosis, these results indicate a direct role of mitochondrial uncoupling in stimulating of apoptosis. Specific suppression of UCP2 also decreased apoptosis while UCP2 overexpression caused the opposite effect. Accordingly, the inhibitory effect of low doses of 1α,25-(OH)₂-D₃ on apoptosis may be mediated by its suppression of UCP2 expression. Although 1α,25-(OH)₂-D₃ increases [C Ca²⁺]i, and such increases are often associated with apoptosis, only the very high dose (>100 nM) of 1,25(OH)2D3 stimulated apoptosis. This result indicates that the pro-apoptotic effect of [Ca²⁺]i with the treatment of 1α,25-(OH)₂-D₃ may be overwhelmed by its anti-apoptotic effect induced by suppression of UCP2.

Glucocorticoid-stimulated apoptosis has been associated with enhanced [Ca²⁺]i influx, providing evidence that increased [Ca²⁺]i might be involved in triggering apoptosis. This hypothesis was further supported by the observation that increased level of inositol 1,4,5-triphosphate(InsP3) receptor in lymphocytes stimulates apoptosis while IP3R-deficient cells are resistant to T-cell receptor (TCR)-induced apoptosis. Calcium was also linked to the release of arachidonic acid from cells triggered to undergo apoptosis by various chemotherapeutic agents, suggesting that calcium is important to many cPLA2-dependent apoptotic responses. Consistent with this, Example 2 confirms that increases in [Ca²⁺]i influx stimulate apoptosis by increasing caspase-3 expression. This result may explain the pro-apoptotic effect of high dose 1α,25-(OH)₂-D₃. In addition, calcium increases precede the cytolysis of the targets of cytotoxic T cells, indicating rapid, sustained [Ca²⁺]i increase is required for the apoptotic process. Although 1α,25-(OH)₂-D₃ induced dose-dependent increases in [Ca²⁺]i, the data in Example 2 indicate that the magnitude of this increases in [Ca²⁺]i may determine whether apoptosis results. It has been previously demonstrated that [Ca²⁺]i storage sites also appear to be affected, as the cytosolic calcium undergo changes in response to apoptotic stimuli. During apoptotic stress, the mitochondrial calcium pool may also be affected, since mitochondrial potential, which plays a key role in maintaining mitochondrial calcium homeostasis, drops very early during apoptotic death. Decreased mitochondrial potential reduces ATP production and causes cytochrome c leakage and mitochondrial swelling. Accordingly, mitochondrial uncoupling might stimulate apoptosis by decreasing mitochondrial potential. This hypothesis is further supported by the observations that the chemical uncoupler DNP decreases adipocyte mitochondrial potential and stimulates apoptosis while in this example, the mitochondrial uncoupling inhibitor GDP causes the opposite effects. Consistent with this, overexpression of UCP2 stimulated apoptotic proteases while suppression of UCP2 decreased expression of these genes, indicating that UCP2 plays a positive role in regulation of apoptosis. 1α,25-(OH)₂-D₃, which has been found to inhibit UCP2 expression via a calcium-independent pathway, suppressed apoptosis at physiological low doses from 0.1 nM to 10 nM, suggesting that the anti-apoptotic effect of low doses of 1α,25-(OH)₂-D₃ might be mediated by suppression of UCP2 expression.

In Example 2, 1α, 25-(OH)₂-D₃ induced a similar effect on [Ca²⁺]i increase as BK 8644 but the two compounds exerted different effects on mitochondrial calcium storage, as low doses 1α,25-(OH)₂-D₃ (0.1 nM to 10 mM) decreased mitochondrial calcium while BK 8644 increased mitochondrial calcium. However, the high dose of 1α,25-(OH)₂-D₃ (100 nM) caused similar increases in mitochondrial calcium as observed in BK 8644. Since mitochondrial calcium overload might trigger apoptosis by inducing mitochondrial potential collapse and cytochrome c release, the pro-apoptotic effect of the high dose of 1α,25-(OH)₂-D₃ is most likely a result of the stimulation of mitochondrial calcium. In contrast, the decreases in mitochondrial calcium observed in response to treatment with lower dose of 1α,25-(OH)₂-D₃ indicates that reduction of mitochondrial calcium load, along with the inhibitory effect on UCP2 contribute to the anti-apoptotic effect of low doses of 1α,25-(OH)₂-D₃.

Although low doses of 1α,25-(OH)₂-D₃ may also induce apoptotic stress via a calcium dependent mechanism, this signal is counter-balanced by the suppression of UCP2, which may increase the capability to maintain intracellular homeostasis by regulating mitochondrial potential and ATP production. Indeed, many calcium channel and pumps located either on plasma membrane or intracellular organelles are ATP dependent. High doses of 1α,25-(OH)₂-D₃, however, induced much greater increases in [Ca²⁺]i, resulting in more rapid and stronger apoptotic signals, which could not be offset by suppression of UCP2.

In summary, Example 2 shows physiological low doses of 1α,25-(OH)₂-D₃ inhibits apoptosis by suppression of UCP2 expression. Since the low doses of 1α,25-(OH)₂-D3 used in this example are within the range of physiological levels which respond to dietary calcium, the anti-obesity effect of high dietary calcium is a result from suppression of apoptosis by 1α,25-(OH)₂-D₃.

From the foregoing description, one skilled in the art can easily ascertain the essential characteristics of this invention, and without departing from the spirit and scope thereof, can make changes and modifications of the invention to adapt it to various usage and conditions.

Without further elaboration, it is believed that one skilled in the art can, using the preceding description, utilize the present invention to its fullest extent. The preceding exemplary specific embodiments are, therefore, to be construed as merely illustrative, and not limitative of the remainder of the disclosure in any way whatsoever.

The entire disclosure of all applications, patents and publications, cited above and below and in the figures are hereby incorporated by reference.

1: A method comprising administering to an individual seeking to reduce adipocytes calcium from a calcium-containing product in an amount effective to induce apoptosis of adipocytes in the individual.

2: The method of claim 1 wherein the calcium from the calcium-containing product is administered as in an amount effective to block calcitrophic hormone (1,25-(OH)₂-D) activity and induce apoptosis in adipocytes in an individual, thereby reducing the amount of adipocytes in said individual.

3-8. (canceled)

9: The method of claim 2, wherein the administering decreases the levels of calcitrophic hormones in the adipocytes.

10: The method of claim 1, wherein the inducing apoptosis comprises killing, depleting, destroying, exterminating, annihilating, eliminating excess of, reducing the number of, or increasing breakdown of fat cells.

11: The method of claim 1, wherein the fat cells are truncal fat cells.

12: The method of claim 1, wherein the number of truncal fat cells is reduced by at least 10%.

13-48. (canceled)

49: The method of claim 1, wherein the calcium from the calcium-containing product modulates one or more of the functional groups of molecules involved in apoptosis.

50: The method of claim 49, wherein the regulated molecule is selected from the caspases or the Bcl-2 family.

51: The method of claim 49, wherein the regulated molecule is a protease.

52: The method of claim 51, wherein the protease is selected from the group consisting of caspase-1, caspase-3 and caspase-9.

53: The method of claim 49, wherein the molecule is a Ca²⁺-dependent endonuclease.

54: The method of claim 50, wherein the Bcl-2 family protein maintains homeostatic concentration of [Ca²⁺]i in endoplasmic reticulum (ER) and mitochondria.

55: The method of claim 50, wherein the Bcl-2 family protein is BAX or BAK.

56: The method of claim 1, wherein the calcium from the calcium-containing product blocks the inhibitory effect of 1α,25-(OH)₂-D₃ on the expression of mitochondrial uncoupling protein 2 (UCP2).

57: The method of claim 1, wherein the calcium from the calcium-containing product induces over-expression of UCP2 leading to marked reductions in mitochondrial potential (ΔΨ) and ATP production, increases in the expression of caspases, and decreases in Bcl-2/Bax expression ratio.

58. (canceled)

59: The method of claim 1, wherein the individual is regulating body weight, inducing weight and/or fat loss, preventing weight and/or fat gain, and/or increasing the metabolic consumption of adipose tissue in the individual.

60-65. (canceled)

66: The method of claim 1, wherein the calcium-containing product is dietary calcium, calcium carbonate, a dairy product and/or a product derived from a dairy product.

67. (canceled)

68. The method of claim 1, wherein the calcium-containing product is a dairy product or derived from a dairy product.

69: The method of claim 68, wherein the dairy product is milk, yogurt or cheese.

70: The method of claim 1, wherein the calcium-containing product comprises a whey-derived protein product that is derived from milk, cream or cheese whey.

71: The method of claim 1, wherein the calcium-containing product is milk or derived from milk.

72: The method of claim 71, wherein the milk is skim milk, 1% milk, 2% milk, or whole milk.

73: The method of claim 1, wherein the calcium-containing product is in the form of a powder, is incorporated into a nutritional or dietary composition or supplement or calcium fortified vitamin supplements, or is incorporated into a food product or foodstuff or is a food high in calcium.

74-75. (canceled)

76: The method of claim 1, wherein the calcium-containing product is added to a food product that is consumed by the individual.

77: The method of claim 76, wherein the food product is a beverage or a liquid supplemented with calcium.

78: The method of claim 76, wherein the food product is selected from the group consisting of acidic juice beverages, acidic beverages, neutral pH beverages, nutritional supplement foodstuffs, confectionery products, dairy products, non-dairy products naturally high in calcium, food or food stuff fortified with calcium, bakery products and farinaceous products.

79: The method of claim 76, wherein the food product is orange juice, apple juice, grape juice, grapefruit juice, cranberry juice, blended juice, milk, soy milk, shake, smoothie, frappe, high-energy protein bar, high calcium chews, chewing gum, chocolate, or cookie, yogurt, frozen yogurt, cheese, processed cheese, bread, muffin, biscuit, cereal or roll.

80. (canceled)

81. The method of claim 1, wherein the calcium-containing product is in the form of a tablet, capsule, or combination with other minerals and/or vitamins.

82. (canceled)

83: The method of claim 1, wherein the calcium-containing product is administered over a prolonged period of time of a continuous interval of at least about two weeks, one month, three months, six months or one year.

84. (canceled)

85: The method of claim 1, wherein the calcium-containing product is administered daily.

86: The method of claim 1, wherein the amount of calcium-containing product effective to induce apoptosis in adipocytes is based on the amount of dietary calcium contained in said product.

87: The method of claim 86, wherein the amount of dietary calcium effective to induce apoptosis in adipocytes is based on the amount of dietary calcium consumed on an average daily basis.

88: The method of claim 87, wherein the average daily amount of consumed dietary calcium effective to induce apoptosis is at least about 500 mg, 600 mg, 700 mg, 800 mg, 900 mg, 1000 mg, 1100 mg, 1200 mg, 1300 mg, 1346 mg, 1400 mg, 1500 mg, 1600 mg, 1700 mg 1800 mg, 1900 mg or 2000 mg.

89: The method of claim 87, wherein the calcium-containing product is administered over a continuous interval and contains at least about 1000 mg on an average daily basis.

90-94. (canceled)

95: The method of claim 1, wherein the individual is on a calorie restricted diet.

96: The method of claim 86, wherein an effective amount of calcium-containing dairy product is at least about 3 servings per day.

97: The method of claim 96, wherein the daily serving is about 6 or 8 ounces of yogurt, 8 ounces of milk or 1.5 ounces of cheese.

98-100. (canceled)

101: The method of claim 96, wherein the serving portion comprises at least about 200, 300, 600, 900 or 1000 mg of dietary calcium.

102-105. (canceled)

106: The method of claim 86, wherein an effective amount of calcium-containing dairy product is least about 60, 90, 100 or 120 servings per month.

107-112. (canceled)
